(19) United States
(12) Patent Application Publication
(10) Pub. No.: US 2013/0331929 A1
(43) Pub. Date: Dec. 12, 2013

(54) Means for Controlled Sealing of Endovascular Devices

(71) Applicant: ENDOLUMINAL SCIENCES PTY LTD., Sydney (AU)

(72) Inventors: Ashish Sudhir Mitra, Sydney (AU); Ben Colin Bobillier, Mosman (AU); Pak Man Victor Wong, Leichhardt (AU)

(21) Appl. No.: 13/844,535

(22) Filed: Mar. 15, 2013

Related U.S. Application Data
(63) Continuation-in-part of application No. 13/476,695, filed on May 21, 2012.
(60) Provisional application No. 61/532,814, filed on Sep. 9, 2011.

(51) Int. Cl. A61L 31/14; A61F 2/24

(52) U.S. Cl. CPC A61L 31/145; A61F 2/24; A61F 2/2427; A61L 31/146
USPC 623/2.11; 623/2.42

(57) ABSTRACT

Expandable sealing means for endoluminal devices have been developed for controlled activation. The devices have the benefits of a low profile mechanism (for both self-expanding and balloon-expanding prostheses), contained, not open, release of the material, active conformation to the “leak sites” such that leakage areas are filled without disrupting the physical and functional integrity of the prosthesis, and on-demand, controlled activation, that may not be pressure activated.

### CROSS-REFERENCE TO RELATED APPLICATIONS

The present application is a continuation-in-part of U.S. Ser. No. 13/476,695, filed May 21, 2012, which claims the benefit of priority to U.S. Ser. No. 61/532,814, filed Sep. 9, 2011, both of which are incorporated herein by reference in their entirety.

### FIELD OF THE INVENTION

The present disclosure is directed generally to endoluminal devices and associated systems and methods, and specifically to a method and devices for controlled actuation of means for sealing of an endoluminal prosthesis to a vessel wall.

### BACKGROUND OF THE INVENTION

An aneurysm is a localized, blood-filled dilation of a blood vessel caused by disease or weakening of the vessel wall. Aneurysms affect the ability of the vessel to conduct fluids, and can be life threatening if left untreated. Aneurysms most commonly occur in arteries at the base of the brain and in the aorta. As the size of an aneurysm increases, there is an increased risk of rupture, which can result in severe hemorrhage or other complications including sudden death. Aneurysms are typically treated by surgically removing a part or all of the aneurysm and implanting a replacement prosthetic section into the body lumen. Such procedures, however, can require extensive surgery and recovery time. Patients often remain hospitalized for several days following the procedure, and can require several months of recovery time. Moreover, the morbidity and mortality rates associated with such major surgery can be significantly high.

Another approach for treating aneurysms involves remote deployment of an endovascular graft assembly at the affected site. Such procedures typically require intravascular delivery of the endovascular graft assembly to the site of the aneurysm. The graft is then expanded or deployed in situ and the ends of the graft are anchored to the body lumen on each side of the aneurysm. In this way, the graft effectively excludes the aneurysm sac from circulation.

One concern with many conventional endovascular graft assemblies, however, is the long term durability of such structures. Over time, the graft can become separated from an inner surface of the body lumen, resulting in bypassing of the blood between the vessel wall and the graft. As used herein, endoleak is defined as a persistent blood or other fluid flow outside the lumen of the endoluminal graft, but within the aneurysm sac or adjacent vascular segment being treated by the device. When an endoleak occurs, it can cause continuous pressurization of the aneurysm sac and may result in an increased risk of rupture.

In addition to endoleaks, another concern with many conventional endovascular graft assemblies is subsequent device migration and/or dislodgement. For example, after a surgeon has found an optimal location for the graft, the device must be fixed to the wall of the body lumen and fully sealed at each end of the graft to prevent endoleaks and achieve a degree of fixation that will prevent subsequent device migration and/or dislodgement.

Aortic stenosis, also known as aortic valve stenosis, is characterized by an abnormal narrowing of the aortic valve. The narrowing prevents the valve from opening fully, which obstructs blood flow from the heart into the aorta. As a result, the left ventricle has to work harder to maintain adequate blood flow through the body. If left untreated, aortic stenosis can lead to life-threatening problems including heart failure, irregular heart rhythms, cardiac arrest, and chest pain. Aortic stenosis is typically due to age-related progressive calcification of the normal trileaflet valve, though other predisposing conditions include congenital heart defects, calcification of a congenital bicuspid aortic valve, and acute rheumatic fever.

For the last fifty years, open heart surgery for aortic valve replacement using cardiopulmonary bypass, sternotomy (or mini-sternotomy), aortic cross clamping and cardioplegic arrest represents the treatment of choice and the standard of care for patients having severe aortic stenosis with symptoms (Bonow, et al., Circulation, 114:e84-231 (2006), Kvidal, et al., J. Am. Coll. Cardiol., 35:747-56 (2000), Otto, Heart, 84:211-8 (2000), Schwarz, et al., Circulation, 66:1105-10 (1982)). However, there is a large pool of patients affected by severe aortic stenosis who are not candidates for open heart valve replacement surgery because they are considered too old (nonagenarians, centenaries) for such an invasive procedure, or because they are also affected by other co-existing conditions that compound their operative risk (Jung, et al., Eur Heart J. 26:2714-20 (2005). For these patients, who are at high surgical risk, a less invasive treatment is necessary.

Transcatheter aortic-valve implantation (TAV) is a procedure in which a bioprosthetic valve is inserted through a catheter and implanted within the diseased native aortic valve. The most common implantation routes include the transapical approach (TA) and transfermoral (TF), though trans-subclavian and trans-aortic routes are also being explored (Ferrari, et al., Swiss Med Wkly, 140:w13127 (2010). These percutaneous routes rely on a needle catheter getting access to a blood vessel, followed by the introduction of a guidewire through the lumen of the needle. It is over this wire that other catheters can be placed into the blood vessel, and implantation of the prosthesis is carried out.

Since 2002 when the procedure was first performed, there has been rapid growth in its use throughout the world for the treatment of severe aortic stenosis in patients who are at high surgical risk, and there is mounting support to adopt the therapy as the standard of care for patients that are not at a high risk for surgery. Clinical studies have shown that the rate of death from any cause at the one-year mark among patients treated with TAV was approximately 25% (Grube, et al., Circ. Cardiovasc. Interv. 1:167-175 (2008), Himbert et al., J. Am. Coll. Cardiol., 54:303-311 (2009), Webb, et al., Circulation, 119:3009-3016 (2009), Rodes-Cabau, et al., J. Am. Coll. Cardiol., 55:1080-1090 (2010), and the results of two parallel prospective, multicenter, randomized, active-treatment-controlled clinical trials showed that TAV is superior to standard therapy, when comparing the rate of death from any cause at the 1-year mark (30.7% in the TAV group, as compared with 50.7% in the standard-therapy group) (Leon, et al., N. Engl. J. Med., 363:1597-1607 (2010)).

Paravalvular leaks are extremely rare in surgical aortic-valve replacement—seen in just 1.5% to 2% of cases. But as experts observed at Euro PCR 2011, mild paravalvular leaks are relatively common in transcatheter aortic-valve implantation (TAV), and new data suggest that more severe paravalvular aortic regurgitation (AR) is a key reason for prosthetic valve dysfunction. According to Dr. Jan-Malte Sinning (Universitatsklinikum, Bonn, Germany), moderate to severe periprosthetic aortic regurgitation occurs in approximately 15% of TAV-treated patients, a number drawn from 12 international registries. In 127 consecutive patients treated with TAV at his center, 21 developed moderate paravalvular AR postprocedure, and this was associated with a significantly higher rate of 30-day and one-year mortality, as well as acute kidney injury, compared with patients with no or mild AR. Predictors of paravalvular AR included a low baseline left ventricular ejection fraction (LVEF) and inadequate sizing of the annulus or device. Dr. Kensuke Takagi (San Raffaele Hospital, Milan, Italy), reported that at his center, 32 patients developed AR grade 2+ to 4+, out of 79 consecutive patients treated with the CoreValve (Medtronic). In multivariate analyses, valve-annulus mismatch, particularly in larger aortic annuli, was a significant predictor of developing more severe paravalvular AR; an even stronger predictor was low implantation of the valve, which increased the risk by more than threefold. And while postdilatation can help treat paravalvular AR, this is appropriate only in patients in whom the valve was correctly positioned at the outset, Takagi said. See Leon M B, Piazza N, Nikolsky E, et al. Standardized endpoint definitions for transcatheter aortic valve implantation clinical trials. J Am Coll Cardiol 2011; 57:253-269; Eur Heart J 2011; 32:205-217

The major potential offered by solving leaks with transcatheter heart valves is in growing the market to the low risk patient segment. The market opportunity in the low-risk market segment is double the size of that in the high risk segment and therefore it is imperative for a TAV device to have technology to provide superior long-term hemodynamic performance so that the physicians recommend TAV over SAVR.

More than 3 million people in the United States suffer from moderate or severe mitral regurgitation (MR), with more than 250,000 new patients diagnosed each year. Functional MR can be found in 84% of patients with congestive heart failure and in 65% of them the degree of regurgitation is moderate or severe. The long term prognostic implications of functional mitral regurgitation have demonstrated a significant increase in risk for heart failure or death, which is directly related to the severity of the regurgitation. Compared to mild regurgitation, moderate to severe regurgitation was associated with a 2.7 fold risk of death and 3.2 fold risk of heart failure, and thus significantly higher health care cost.

Treatment of mitral valve regurgitation depends on the severity and progression of signs and symptoms. Left unchecked, mitral regurgitation can lead to heart enlargement, heart failure and further progression of the severity of mitral regurgitation. For mild cases, medical treatment may be sufficient. For more severe cases, heart surgery might be needed to repair or replace the valve. These open-chest/open-heart procedures carry significant risk, especially for elderly patients and those with severe co-morbidities. While several companies are attempting to develop less invasive approaches to repair the mitral valve, they have found limited anatomical applicability due to the heterogeneous nature of the disease and, so far, have had a difficult time demonstrating efficacy that is equivalent to surgical approaches. Innovative approaches to less invasive heart valve replacement are a promising alternative and Transcatheter Mitral Valve Implantation (TMVI) devices are under development. PVL is likely to be a major problem with these devices and more critical than it is in the case of TAV devices. This is in part due to the lesser degree of calcification observed at the mitral valve replacement site, requiring the device have greater holding power.

TAV and TMVI devices may also be used to treat the disease states of aortic insufficiency (or aortic regurgitation) and mitral stenosis respectively, which are less prevalent compared to the aforementioned valvular disease states, yet have similar or worse clinical prognosis/severity. They can also be implanted within failing bioprostheses that are already implanted surgically, referred to as a valve-in-valve procedure.

An improved device for treatment of these conditions has been developed which includes a means for sealing the device at the site of placement, using a sealing ring that is activated by pressure as it is expanded in situ. As the device expands, a swellable material is released into the sealing means that causes the sealing means to expand and conform to the vessel walls, securing it in place. See WO2010/083558 by Endoluminal Sciences Pty Ltd. The mechanical constraints of these seals are extremely difficult to achieve—require rapid activation in situ, sufficient pressure to secure but not to deform or displace the implanted prosthesis, biocompatibility, and retention of strength and flexibility in situ over a prolonged period of time.

It is therefore an object of the present invention to provide improved physician controllable means for sealing endovascular devices such as stents and aortic valves in situ.

It is a further object of the present invention to provide means for active conformation of the sealing means to the vascular anatomy if any remodeling occurs after implantation so that any resulting leaks are sealed.

It is a further object of the present invention to provide sealing means to support fixation, anchoring or landing platform of/for the TAV device, especially in individuals lacking sufficient calcification in the native valve and in individual with aortic insufficiency as a diseased state.

It is a further object of the present invention to provide expandable materials, such as hydrogels, with the appropriate chemical and physical properties to permanently seal an endoluminal device to a vessel wall.

### SUMMARY OF THE INVENTION

Expandable sealing means for endoluminal devices have been developed for controlled activation. These include a means for controlled activation at the site where the device is to be secured, which avoid premature activation that could result in misplacement or leakage at the site. The sealing means for placement at least partially between an endoluminal prosthesis and a wall of a body lumen has a first relatively reduced radial configuration and a second relatively increased radial configuration which is activated by exposure of a hydratable material within the seal, such as a hydrogel, foam or sponge, for example, by removal of a laminate around the hydrateable seal or by opening of valve thereby allowing liquid to reach the swellable material. Swelling upon contact with fluid at the site expands the sealing means into secure contact with the lumen walls. A semi-permeable membrane is used to prevent the hydrogel gel material from escaping the seal, yet allows access of the fluid to the hydrogel. In preferred embodiments, the swellable material is spray dried onto the interior of the seal, optionally tethered to the material chemically by covalent crosslinking. This material typically has a permeability in the range of five to 70 microns, most preferably 35 to allow rapid access of the fluid to the hydrogel. The sealing means is particularly advantageous since it expands into sites to eliminate all prosthetic-annular incongruities, as needed. A major advantage of these devices is that the sealing means creates little to no increase in profile, since it remains flat/inside or on the device until the sealing means is activated.

Exemplary endoluminal devices including the sealing means for controlled activation include stents, stent grafts for aneurysm treatment and transcutaneously implanted aortic valves (TAV) or mitral, tricuspid or pulmonary valves. In all embodiments, the sealing means is configured to maintain the same low profile as the device without the sealing means. In a preferred embodiment, the sealing means is positioned posterior to the prosthetic implant, and is expanded or pulled up into a position adjacent to the implant at the time of placement/deployment or sealing. This is achieved using sutures or elastic means to pull the seal up and around the implant at the time of placement, having a seal that expands up around implant, and/or crimping the seal so that it moves up around implant when the implant comes out of introducer sheath. This is extremely important with large diameter implants such as aortic valves, which are already at risk of damage to the blood vessel walls during transport. In another embodiment, the seal is placed around the skeleton of the TAV, so that it expands with the skeleton at the time of implantation of the TAV. In a variation of this embodiment, the seal is placed between the TAV and the skeleton, and expands through the skeleton sections at the time of implantation to insure sealing.

In all embodiments, it is absolutely critical that the hydrogel/expandable material operates under sufficient low pressure so that it does not push the stent away from the wall or alter the device configuration. These materials must expand quickly (less than ten minutes, more preferably less than five minutes to full swelling), expand to a much greater volume (from two to 100 fold, more preferably from 50 to 90 fold, most preferably sixty fold), and retain the desired mechanical and physiochemical properties for an extended period of time, even under the stress of being implanted with the vasculature or heart. Gels having the desired mechanical and swellable properties have been developed, as demonstrated by the examples.

These devices have the advantages of providing excellent sealing in combination with a low profile, controlled or contained release, and active conforming to leak sites to eliminate prosthetic-annular incongruence. If vascular re-modeling occurs over time, which could lead to leakage, the seal will also remodel, preventing leaks from developing. For devices that are at high risk of leakage, a pleated or accordion-like design provides for even better coverage and prevents uneven distribution of seal filler.

### BRIEF DESCRIPTION OF THE DRAWINGS

FIGS. 1A, 1B and 1C are perspective views of a transcatheter aortic valve (TAV) (FIG. 1A), a controlled activatable seal (FIG. 1B), and the seal placed around the TAV (FIG. 1C).

FIGS. 2A, 2B and 2C are perspective views of the TAV of FIG. 1C crimped toward the inflow side of the TAV in a telescopic manner (FIG. 2A), with the TAV and seal in an expanded state with the stent aligned with the bottom section of the TAV, with the activation wire activated to expose the seal to fluids (FIG. 2B), and post deployment, with the seal expanded by swelling of the hydrogel within the seal when it contacts the blood.

FIG. 3 is a perspective cross-sectional view of the seal, showing the inner and outer membranes, hydrogel within the inner membrane and the activation site.

FIGS. 4A-4D are schematics of a teardrop capsule. FIG. 4A is a perspective view showing the film made of a polymeric material such as polyetheretherketone (PEEK), polyethylene terephthalate (PET) or polyurethane (PU); heat sealed, laser welded, seal; hydrogel strip; and mesh; FIG. 4B is a perspective view of the assembly of the film, hydrogel and seal; FIG. 4C is a perspective view showing the film positioned on the exterior of an expanded TAV; and FIG. 4D is a cross-sectional view showing the opening slit from the top to allow for hydration of the hydrogel strip during diastole.

FIGS. 4E and 4F are cross-sectional views of the teardrop capsule of FIGS. 4A-4D. FIG. 4E shows the film overlaying the mesh, having the hydrogel strip positioned thereon, overlaid by the sealed film.

FIGS. 5A-5D are perspectives of an Ice bag seal (FIGS. 5A. 10B), and in cross-section (FIGS. 5C, 5D) showing hydration of the hydroseal when blood pours in ((FIG. 5C), then the opening closes when the hydrogel swells (FIG. 5D).

FIGS. 6A-6D are perspective views of D profile capsule, showing the blow molded D balloon formed by the film sealed over the hydrogel strip positioned on the mesh (FIGS. 6A, B), and the assembly of the TAV device with seal shown in FIGS. 6C and 6D.

FIGS. 7A-7D are perspective views of the TAV in the stent (FIG. 7A), the TAV expanded (FIG. 7B), the TAV expanded and pulled back with the capsule seal flipped over (FIG. 7C), and the TAV and seal expanded (FIG. 7D).

FIG. 8A is a cross-sectional view of a TAVI stent with a flippable strap in a catheter with a HG capsule within the TAV, that flips over onto the outside of the TAVE, after the balloon is inflated to center the TAV. FIG. 8B is a cross-sectional view of the TAVI stent with capsule after struts flip over when the catheter is pulled back; showing the balloon inflation centering the catheter. FIG. 8C is a cross-sectional view of the capsule sitting on the outside of stent, which can be retrieved into the catheter if needed.

FIGS. 9A-9B are perspective (FIG. 9A) and cross-sectional (FIG. 9B) view of the O-ring seal, showing a U shaped casing that encapsulates the seal assembly during storage, preventing hydration of hydrogel by preservative, such as glutaraldehyde.

FIGS. 10A and 10B are perspective and cross-sectional views, respectively, of a foam seal, which is attached to the inside of TAV struts so that the foam is forced through the struts and into leak sites using spring struts (FIG. 10A) or using a balloon.

FIG. 11 is a perspective view of a TAV with a dissolvable film to seal the capsule to prevent hydration.

FIGS. 12A-12E are perspective views of a pre-cut, molded solid silicone core (FIG. 12A) that sits inside of the valve (FIG. 12B) with the metal struts sitting flush within the recesses (FIG. 12C), wherein the seal capsule is on the outside or inside of the frame (FIG. 12D) showing the maximum height of the silicone core to allow for suturing on top part; and the TAV with a silicon sleeve placed over the frame and capsule assembly, sandwiching the stent frame and capsule by virtue of the elastic properties of the band and mechanical pressure from the ratchet mechanism (FIG. 12E).

FIGS. 13A-13D are perspective views of a Metronics TAV with a metal polymer laminate surrounding the capsule, heat sealed in front and back (FIG. 13A), with the tab pulled around the stent frame breaking the heat seal bond and the bottom pull tables pulled to remove the protective cover to prevent hydration during storage (FIG. 13B), shown in cross-section in FIG. 13C, and completely removed as shown in FIG. 13D.

FIGS. 13E-13F show the device of FIGS. 13A-13D, with the remainder of the metal-polymer film pulled away from the capsule via the bottom pull tab (FIG. 13E), detaching the protective covering completely (FIG. 13F), leaving the sealed TAV separate from the covering (FIG. 13G).

FIG. 14 is a cross-sectional view of the metal laminate of FIG. 13.

FIGS. 15A-15D are perspective (FIGS. 15A, 15B) and cross-sectional (FIGS. 15C, 15D) views of a packaging case.

FIG. 16 is a cross-sectional view of a package for a stent with silicone core and ratchet band which is placed into a cap of a liquid silicone.

FIG. 17 is a cross-sectional view of a package includeing a tapered jar and compression disc to separate the liquid around the TAV from the hydratable seal.

FIG. 18 is a package showing a cotton ball on the tissue to protect the seal during storage.

### DETAILED DESCRIPTION OF THE INVENTION

### I. Definitions

“Hydrogel” refers to a substance formed when an organic polymer (natural or synthetic) is crosslinked via covalent, ionic, or hydrogen bonds to create a three-dimensional open-lattice structure which entraps water molecules to form a gel.

“Biocompatible” generally refers to a material and any metabolites or degradation products thereof that are generally non-toxic to the recipient and do not cause any significant adverse effects to the subject.

“Biodegradable” generally refers to a material that will degrade or erode by hydrolysis or enzymatic action under physiologic conditions to smaller units or chemical species that are capable of being metabolized, eliminated, or excreted by the subject. The degradation time is a function of material composition and morphology.

As used herein, “rapidly” expanding refers to a material which reaches its desired dimensions in less than ten minutes after activation or exposure to fluid, more preferably in less than five minutes.

### II. Endoluminal Device Seal

A. Endoluminal Devices

Endoluminal prosthesis and sealing devices are advanced through a body lumen in a first undeployed and reduced profile configuration. When positioned in situ, the sealing device expands from its reduced radial profile configuration to a second configuration with an increased radial profile. In situ, and in its second configuration, the sealing device is configured to be positioned between the prosthesis and the wall of the body lumen. In one embodiment, when the endoluminal prosthesis is at the desired location in the body lumen, it is typically deployed from an introducer catheter whereupon it may move to an expanded radial configuration by a number of mechanisms. In some embodiments, the prosthesis may be spring expandable. Alternatively, a balloon or expandable member can be inflated within the lumen of the prosthesis to cause it to move to an expanded radial configuration within the vessel. This radial expansion, in turn, presses the sealing device against a wall of the body lumen. One of the advantages of the seal is that it only fills the gaps, and does not impact the placement and integrity—both physical and functional, of the prosthetic or the implant.

In one embodiment, the sealing device is configured to fully seal a proximal, central and/or distal end of the endoluminal prosthesis for endovascular aneurysm repair (EVAR) to prevent endoleaks and prevent subsequent migration and/or dislodgement of the prosthesis.

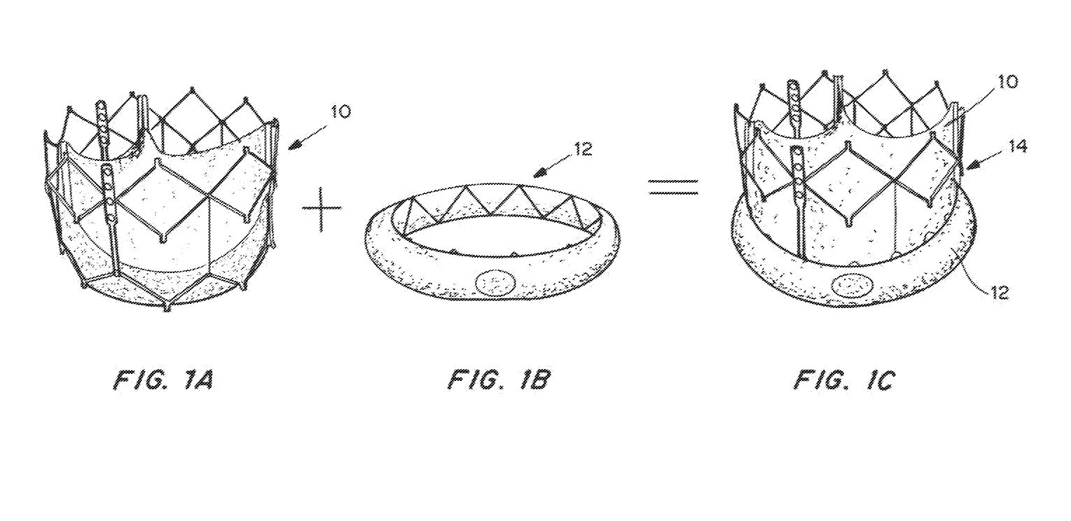

In another embodiment, the sealing device is configured to fully seal a transcatheter aortic valve. FIGS. 1A, 1B and 1C are perspective views of a transcatheter aortic valve (TAV) 10 (FIG. 1A), a controlled activatable seal (FIG. 1B) 12, and the seal placed around the TAV 14 (FIG. 1C).

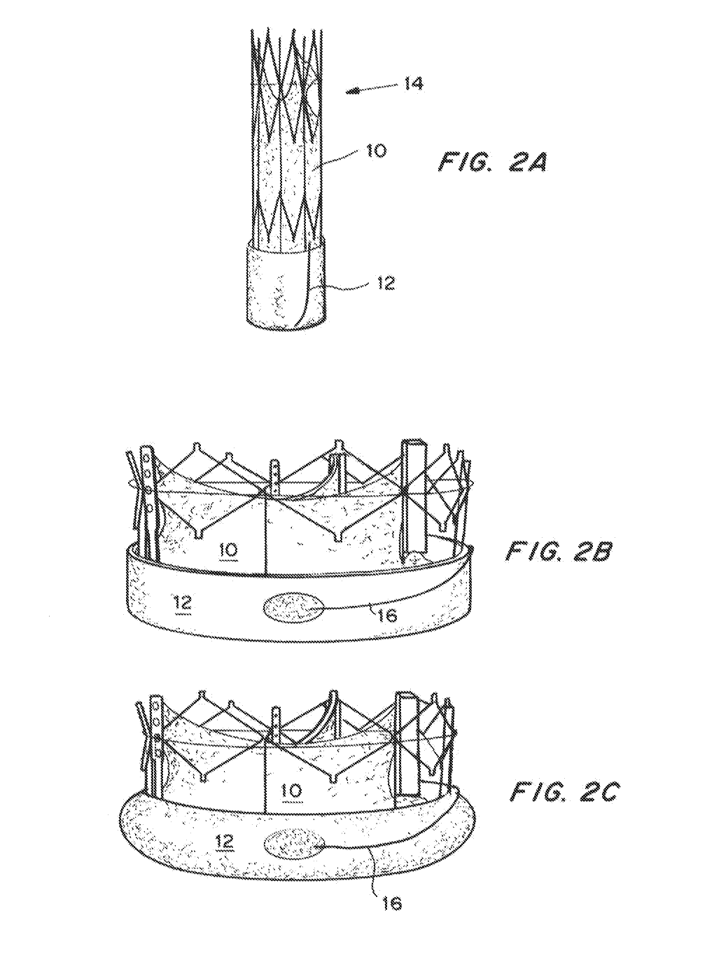

FIGS. 2A, 2B and 2C are perspective views of the TAV 14 of FIG. 1C crimped toward the inflow side of the TAV 10 in a telescopic manner (FIG. 2A), with the TAV 10 and seal 12 in an expanded state with the stent aligned with the bottom section of the TAV, with the activation wire 16 activated to expose the seal 12 to fluids (FIG. 2B), and post deployment, with the seal 12 expanded by swelling of the hydrogel within the seal when it contacts the blood.

The endoluminal device may be configured such that it moves independently of the endoluminal prosthesis. Alternatively, the endoluminal device may be connected to the prosthesis for delivery to a target site. The endoluminal device may be connected to the prosthesis by any number of means including suturing, crimping, elastic members, magnetic or adhesive connection.

In one embodiment, the sealing means is positioned posterior to the prosthetic implant, and is expanded and pulled up into a position adjacent to the implant at the time of sealing. This is achieved using sutures or elastic means to pull the seal up and around the implant at the time of placement, having a seal that expands up around implant, and/or crimping the seal so that it moves up around implant when implant comes out of introducer sheath. This is extremely important with large diameter implants such as aortic valves, which are already at risk of damage to the blood vessel walls during transport.

A key feature of the latter embodiment of the seal technology is that it enables preservation of the crimped profile of the endoluminal prosthesis. The seal technology is positioned distal or proximal to the prosthesis. In one aspect of this technology, the seal is aligned with the prosthesis by expansion of the seal. In another aspect, the seal zone of the prosthesis is aligned with the seal zone prior to expansion of the prosthesis. In additional embodiments, the seal is positioned between the device skeleton and the device, or on the exterior of the skeleton.

In a further embodiment, the endoluminal device may further include one or more engagement members. The one or more engagement members may include staples, hooks or other means to engage with a vessel wall, thus securing the device thereto.

B. The Seal

The seal includes a flexible component that is configured to conform to irregularities between the endoluminal prosthesis and a vessel wall. The seal includes a generally ring-like structure having a first or inner surface and a second or outer surface. It contains a material that swells upon contact with a fluid or upon activation of a foam, following placement, to inflate and conform the seal around the device.

The seal can be provided in a variety of shapes, depending on the device it is to be used with. A “D” shape is the preferred embodiment, with the flat portion being attached to the support structure and/or device to be implanted.

The seal can be composed of a permeable, semi-permeable, or impermeable material. It may be biostable or biodegradable. For example, the seal may be composed of natural or synthetic polymers such as polyether or polyester polyurethanes, polyvinyl alcohol (PVA), silicone, cellulose of low to high density, having small, large, or twin pore sizes, and having the following features: closed or open cell, flexible or semi-rigid, plain, melamine, or post-treated impregnated foams. Additional materials for the seal can include polyvinyl acetal sponge, silicone sponge rubber, closed cell silicone sponges, silicone foam, and fluorosilicone sponge. Specially designed structures using vascular graft materials including polytetrafluoroethylene (PTFE), polyethylterephthalate (PET), polyether ether ketone (PEEK), woven yarns of nylon, polypropylene (PP), collagen or protein based matrix may also be used. PEEK is the preferred material at this time since the strength is high so that there will be no damage leading to failure when the TAV device is expanded against sharp/calcified nodules and at the same time a relatively thin sheet of material can be used, helping maintain a lower profile.

The seal material may be used independently or in combination with a mesh made from other types of polymers, titanium, surgical steel or shape memory alloys.

The capsule may include an outer wall to hold the agent therein. The outer wall may be made of a suitably flexible and biocompatible material. Alternatively, the capsule may include a more rigid structure having a pre-designed failure mechanism to allow the release of agent therefrom. Examples of suitable materials include, but are not limited to, low density polyethylene, high density polyethylene, polypropylene, polytetrafluoroethylene, silicone, or fluorosilicone. Other fluoropolymers that may be used for the construction of the capsule include: polytetrafluoroethylene, perfluoroalkoxy polymer resin, fluorinated ethylene-propylene, polyethylenetetrafluoroethylene, polyvinylfluoride, ethylenechlorotrifluoroethylene, polyvinylidene fluoride, polylychlorotrifluoroethylene, perfluoropolyether, fluorinated ethylene propylene, terpolymer of tetrafluoroethylene, hexafluoropropylene and vinylidene fluoride), polysulphone and polyether ether ketone (PEEK). It may also include non-polymeric materials such as glass, bioglass, ceramic, platinum and titanium. It may further include biologically based materials such as crosslinked collagen or alginates. It will be appreciated that the foregoing list is provided merely as an example of suitable materials and is not an exhaustive list. The capsule may be composed of a material or combination of materials different from those provided above.

The rate of release of the agent from the support member may vary. In some embodiments, pressure exerted on the support member to rupture a capsule may release one or more agents. This rate of almost immediate release is particularly useful for delivering adhesive agents to a vessel to affix a prosthesis to a wall of the vessel. However, other agents may be released at a slower or at least a variable rate. Further, the agents may be released after the initial release of a primary agent (e.g. the adhesive).

A variety of different techniques or processes can be used to form pressure activated capsules or compartments. In one embodiment, for example, a process for forming a pressure activated capsule includes pre-stressing the capsule during formation. The pre-stressed material will have a limited capacity to stretch when subjected to external pressure, and will fail when reaching critical stress on the stress-strain curve. The first stage of this method includes selecting a biocompatible capsule material that is also compatible with its contents (e.g., the agent which can include adhesive material or a wide variety of other types of materials). The capsule material should also have a tensile strength suitable for the particular application in which the capsule will be used.

Permeable and Impermeable Membranes

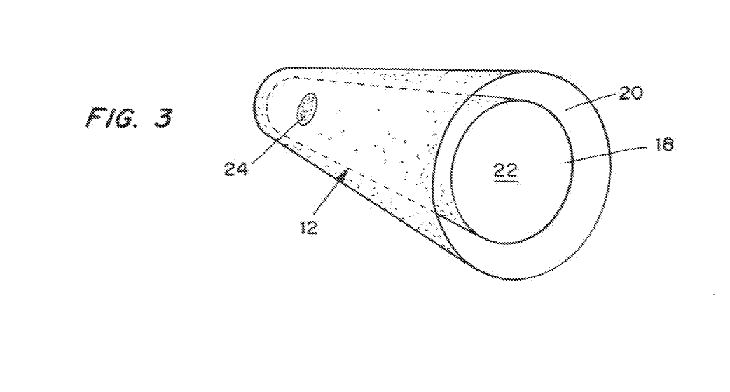

In one embodiment, shown in FIG. 3, the seal 12 includes two membranes, an inner membrane 18 and an outer membrane 20. An expandable material such as a foam or hydrogel 22 is placed within the inner membrane 18. The inner membrane 18 is semi-permeable (allowing fluid ingress but not egress of entrapped hydrogel or foam) while the outer membrane 20 is impermeable except at an optional pre-determined opening 24. The outer membrane 20 is designed to be impermeable to fluid during storage and transport and during any pre-procedural preparations e.g. rinsing or washing of the device, to protect the polymer 22 from premature swelling. The outer membrane 20 is also designed to be strong and puncture resistant so that it does not tear or is punctured or pierced by the sharp edges of the native calcification even when subject to pressures up to 14 atm. This prevents the rupture of the inner membrane 18, mitigating any risk of embolization of the expandable material or hydrogel 22. The rupture point 24 allows fluid such as blood to penetrate into the expandable seal only when the seal is expanded in place, thereby preventing leaks.

Permeable membranes may be made from a variety of polymer or organic materials, including polyimides, phospholipid bilayer, thin film composite membranes (TFC or TFM), cellulose ester membranes (CEM), charge mosaic membranes (CMM), bipolar membranes (BPM), and anion exchange membranes (AEM).

A preferred pore size range for allowing fluid in but not hydrogel to escape is from five to seventy microns, more preferably about 35 to seventy microns, most preferably about 35 microns, so that the fluid can rapidly access the swellable material.

The permeable membrane may be formed only of permeable material, or may have one or more areas that are impermeable. This may be used to insure that swelling does not disrupt the shape of the seal in an undesirable area, such as on the interior of the device where it abuts the implant or prosthesis, or where it contacts the device support members.

In some embodiments, the second impermeable membrane is applied with plasma vapour deposition, vacuum deposition, co-extrusion, or press lamination.

Expandable Materials

Expandable materials which swell in contact with an aqueous fluid are preferred. Most preferably, these materials expand from two to 100 times; more preferably from 50 to 90 fold, most preferably about 60 fold. Blood and/or other fluids at the site of implantation can penetrate into the seal after it is breached, causing dried or expandable materials to absorb the fluid and swell or react to expand due to formation or release of gas reaction products. The semi-permeable inner membrane prevents the expandable material from escaping the seal, but allows fluid to enter. By expanding in volume, the material seals the endoluminal space.

Any expandable material having suitable physical and chemical properties may be used. In certain embodiments, the expandable material is a hydrogel. Other suitable materials include foams and sponges formed at the time of activation.

Expandable materials are chosen to be stable at both room temperature and 37-40° C. and to be sterilizable by one or more means such as radiation or steam. Sponges or foams can be made from biocompatible materials that allow tissue ingrowth or endothelialisation of the matrix. Such endothelialisation or tissue ingrowth can be facilitated either through selection of appropriate polymeric materials or by coating of the polymeric scaffold with suitable growth promoting factors or proteins.

1. Hydrogels

The properties of the hydrogel are selected to provide a rapid swell time as well as to be biocompatible in the event of a breach of capsule integrity. Two or more hydrogels or other materials that swell may be used.

Expandable gels have been developed that are stronger and more resilient than current expandable gels. These gels are able to expand rapidly to at least 10× the dry state and more preferably up to 50× their dry state when exposed to physiological liquids. These stronger gels are synthesized using long chain cross-linkers, typically molecules with more than 20 carbon atoms and/or a molecular weight greater than 400 Da, more preferably more than 40 carbon atoms and/or a molecular weight greater than 800 Da, that will act as molecular reinforcement molecules, creating a more resilient and longer lasting gel while maintaining excellent swelling properties. The swelling force of these gels can also be adjusted to not exert more radial force than necessary, typically around 0.001N/mm² to 0.025N/mm². An ideal range is 0.008N/mm² to 0.012N/mm².

In some embodiments, these gels can be spray dried or chemically attached to a base membrane or mesh used to encapsulate the gel before being fitted to the surgical device. This can be done by attaching either allylic, vinyl or acrylic groups. An allyl group is a substituent with the structural formula H2C═CH—CH2R, where R is the connection to the rest of the molecule. It is made up of a methylene (—CH2-), attached to a vinyl group (—CH═CH₂). An acrylic group includes an acryloyl group has the structure H₂C═CH—C(═O)—; it is the acyl group derived from acrylic acid. The preferred IUPAC name for the group is prop-2-enoyl, and it is also (less correctly) known as acrylyl or simply acryl. Compounds containing an acryloyl group can be referred to as “acrylic compounds”. A vinyl compound (formula —CH═CH₂) is any organic compound that contains a vinyl group (Preferred IUPAC name ethenyl), which are derivatives of ethene, CH₂═CH₂, with one hydrogen atom replaced with some other group to the base substrate, either as small molecules or as long chain tentacles. Long chain hydrophilic polymers useful as described herein with more than 20 atoms in a chain and/or a molecular weight greater than 400 Da, more preferably more than 40 atoms in a chain and/or a molecular weight greater than 800 Da, which have at least two and preferably more than two reactive groups capable of participating in a free radical polymerization reaction and where at least part of the molecule is attached to a substrate, anchoring the gel to the substrate to prevent release of smaller gel particles in case of gel fracture. Long-chain cross-linkers and/or the chemical attachment of the gels to a porous substrate will result in gels that are more capable of withstanding cyclic loads. These seals containing gels can be made in any shape, including annular or strip shape.

The principle behind these cross-linkers is that rather than having a short cross-linker with only two polymerizable groups, a type includes long chain hydrophilic polymer (examples are PVA, PEG, PVAc, natural polysaccharides such as dextran, HA, agarose, and starch)) of long-chain hydrophilic polymer with multiple polymerizable groups is used. The benefits are a much stronger hydrogel, approximately 0.001N/mm² to 0.025N/mm², more preferably between 0.008N/mm² to 0.012N/mm², as compared to hydrogels crosslinked with short chain divalent linkers, as noted above, less than 20 carbon atoms and/or a molecular weight of less than 400 Da with two active groups that can be used for cross-linking (e.g. vinyl, acrylic, allylic)). Interestingly, while these gels are very firm, they at the same time possess very good swelling characteristics. Very strong gels do not swell as much and/or as rapidly. As used herein, very strong refers generally to hydrogels having a strength greater than about 0.001N/mm² to 0.025N/mm². Desired rates of swelling are 30× or greater, with an ideal range of 50×-80×. The greater the swelling rate, the smaller the introduction profile of the device, allowing treatment of a greater number of patients who have smaller access vessels (femoral arteries, radial arteries, etc.)).

Suitable components of such gels include, but are not limited to, acrylic acid, acrylamide or other polymerizable monomers; cross-linkers such as polyvinyl alcohols as well as partially hydrolyzed poly vinyl acetates, 2-hydroxyethyl methacrylates (HEMA) or various other polymers with reactive side groups such as acrylic, allylic, and vinyl groups, can be used. In addition, a wide range of natural hydrocolloids such as dextran, cellulose, agarose, starch, galactomannans, pectins, hyaluronic acid etc. can be used. Reagents such as allyl glycidyl ether, allyl bromide, allyl chloride etc. can be used to incorporate the necessary double bonds to participate in a free radical polymerization reaction, such as those containing acrylic, allylic and vinyl groups, into the backbones of these polymers. Depending on the chemistry employed, a number of other reagents can be used to incorporate reactive double bonds.

Studies to identify hydrogels having substantial swelling in a short time were performed, as described in examples 1 and 2. The main factors that influence swelling of a hydrogel based on polymerisation and cross-linking of synthetic monomers are:

(1) type of monomer;
(2) type of cross-linker;
(3) concentration of monomer and cross-linker in the gel; and
(4) the ratio of monomer to cross-linker.

Examples of rapidly swelling hydrogels include, but are not limited to, acrylic acid polymers and copolymers, particularly crosslinked acrylic acid polymer and copolymers. Suitable crosslinking agents include acrylamide, di(ethylene glycol) diacrylate, poly(ethylene glycol) diacrylate, and long-chain hydrophilic polymers with multiple polymerizable groups, such as poly vinyl alcohol (PVA) derivatized with allyl glycidyl ether. Additional examples of materials which can be used to form a suitable hydrogel include polysaccharides such as alginate, polyphosphazines, poly(acrylic acids), poly(methacrylic acids), poly(alkylene oxides), poly(vinyl acetate), polyvinylpyrrolidone (PVP), and copolymers and blends of each. See, for example, U.S. Pat. Nos. 5,709,854, 6,129,761 and 6,858,229.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups and sulfonic acid groups.

Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

A water-soluble gelling agent such as a polysaccharide gum, more preferably a polyanionic polymer like alginate, can be cross-linked with a polycationic polymer (e.g., an amino acid polymer such as polylysine) to form a shell. See e.g., U.S. Pat. Nos. 4,806,355, 4,689,293 and 4,673,566 to Goosen et al.; U.S. Pat. Nos. 4,409,331, 4,407,957, 4,391,909 and 4,352,883 to Lim et al.; U.S. Pat. Nos. 4,749,620 and 4,744,933 to Rha et al.; and U.S. Pat. No. 5,427,935 to Wang et al. Amino acid polymers that may be used to crosslink hydrogel forming polymers such as alginate include the cationic poly(amino acids) such as polylysine, polyarginine, polyornithine, and copolymers and blends thereof.

Other exemplary polysaccharides include chitosan, hyaluronan (HA), and chondroitin sulfate. Alginate and chitosan form crosslinked hydrogels under certain solution conditions, while HA and chondroitin sulfate are preferably modified to contain crosslinkable groups to form a hydrogel. Alginate forms a gel in the presence of divalent cations via ionic crosslinking. Although the properties of the hydrogel can be controlled to some degree through changes in the alginate precursor (molecular weight, composition, and macromer concentration), alginate does not degrade, but rather dissolves when the divalent cations are replaced by monovalent ions. In addition, alginate does not promote cell interactions. See U.S. Pat. No. 4,391,909 to Lim et al. for description of alginate hydrogel crosslinked with polylysine. Other cationic polymers suitable for use as a cross-linker in place of polylysine include poly(β-amino alcohols) (PBAAs) (Ma M, et al. Adv. Mater. 23:H189-94 (2011).

Chitosan is made by partially deacetylating chitin, a natural nonmammalian polysaccharide, which exhibits a close resemblance to mammalian polysaccharides, making it attractive for cell encapsulation. Chitosan degrades predominantly by lysozyme through hydrolysis of the acetylated residues. Higher degrees of deacetylation lead to slower degradation times, but better cell adhesion due to increased hydrophobicity. Under dilute acid conditions (pH<6), chitosan is positively charged and water soluble, while at physiological pH, chitosan is neutral and hydrophobic, leading to the formation of a solid physically crosslinked hydrogel. The addition of polyol salts enables encapsulation of cells at neutral pH, where gelation becomes temperature dependent.

Chitosan has many amine and hydroxyl groups that can be modified. For example, chitosan has been modified by grafting methacrylic acid to create a crosslinkable macromer while also grafting lactic acid to enhance its water solubility at physiological pH. This crosslinked chitosan hydrogel degrades in the presence of lysozyme and chondrocytes. Photopolymerizable chitosan macromer can be synthesized by modifying chitosan with photoreactive azidobenzoic acid groups. Upon exposure to UV in the absence of any initiator, reactive nitrene groups are formed that react with each other or other amine groups on the chitosan to form an azo crosslink.

Hyaluronan (HA) is a glycosaminoglycan present in many tissues throughout the body that plays an important role in embryonic development, wound healing, and angiogenesis. In addition, HA interacts with cells through cell-surface receptors to influence intracellular signaling pathways. Together, these qualities make HA attractive for tissue engineering scaffolds. HA can be modified with crosslinkable moieties, such as methacrylates and thiols, for cell encapsulation. Crosslinked HA gels remain susceptible to degradation by hyaluronidase, which breaks HA into oligosaccharide fragments of varying molecular weights. Auricular chondrocytes can be encapsulated in photopolymerized HA hydrogels where the gel structure is controlled by the macromer concentration and macromer molecular weight. In addition, photopolymerized HA and dextran hydrogels maintain long-term culture of undifferentiated human embryonic stem cells. HA hydrogels have also been fabricated through Michael-type addition reaction mechanisms where either acrylated HA is reacted with PEG-tetrathiol, or thiol-modified HA is reacted with PEG diacrylate.

Chondroitin sulfate makes up a large percentage of structural proteoglycans found in many tissues, including skin, cartilage, tendons, and heart valves, making it an attractive biopolymer for a range of tissue engineering applications. Photocrosslinked chondroitin sulfate hydrogels can be been prepared by modifying chondroitin sulfate with methacrylate groups. The hydrogel properties were readily controlled by the degree of methacrylate substitution and macromer concentration in solution prior to polymerization. Further, the negatively charged polymer creates increased swelling pressures allowing the gel to imbibe more water without sacrificing its mechanical properties. Copolymer hydrogels of chondroitin sulfate and an inert polymer, such as PEG or PVA, may also be used.

Biodegradable PEG hydrogels can be been prepared from triblock copolymers of poly(α-hydroxy esters)-b-poly (ethylene glycol)-b-poly(α-hydroxy esters) endcapped with (meth)acrylate functional groups to enable crosslinking PLA and poly(8-caprolactone) (PCL) have been the most commonly used poly(α-hydroxy esters) in creating biodegradable PEG macromers for cell encapsulation. The degradation profile and rate are controlled through the length of the degradable block and the chemistry. The ester bonds may also degrade by esterases present in serum, which accelerates degradation. Biodegradable PEG hydrogels can also be fabricated from precursors of PEG-bis-[2-acryloyloxy propanoate]. As an alternative to linear PEG macromers, PEG-based dendrimers of poly(glycerol-succinic acid)-PEG, which contain multiple reactive vinyl groups per PEG molecule, can be used. An attractive feature of these materials is the ability to control the degree of branching, which consequently affects the overall structural properties of the hydrogel and its degradation. Degradation will occur through the ester linkages present in the dendrimer backbone.

The biocompatible, hydrogel-forming polymer can contain polyphosphoesters or polyphosphates where the phosphoester linkage is susceptible to hydrolytic degradation resulting in the release of phosphate. For example, a phosphoester can be incorporated into the backbone of a crosslinkable PEG macromer, poly(ethylene glycol)-di-[ethylphosphatidyl (ethylene glycol) methacrylate] (PhosPEG-dMA), to form a biodegradable hydrogel. The addition of alkaline phosphatase, an ECM component synthesized by bone cells, enhances degradation. The degradation product, phosphoric acid, reacts with calcium ions in the medium to produce insoluble calcium phosphate inducing autocalcification within the hydrogel. Poly(6-aminoethyl propylene phosphate), a polyphosphoester, can be modified with methacrylates to create multivinyl macromers where the degradation rate was controlled by the degree of derivitization of the polyphosphoester polymer.

Polyphosphazenes are polymers with backbones consisting of nitrogen and phosphorous separated by alternating single and double bonds. Each phosphorous atom is covalently bonded to two side chains. The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca²⁺ or Al³⁺. Polymers can be synthesized that degrade by hydrolysis by incorporating monomers having imidazole, amino acid ester, or glycerol side groups. Bioerodible polyphosphazines have at least two differing types of side chains, acidic side groups capable of forming salt bridges with multivalent cations, and side groups that hydrolyze under in vivo conditions, e.g., imidazole groups, amino acid esters, glycerol and glucosyl. Hydrolysis of the side chain results in erosion of the polymer. Examples of hydrolyzing side chains are unsubstituted and substituted imidizoles and amino acid esters in which the group is bonded to the phosphorous atom through an amino linkage (polyphosphazene polymers in which both R groups are attached in this manner are known as polyaminophosphazenes). For polyimidazolephosphazenes, some of the “R” groups on the polyphosphazene backbone are imidazole rings, attached to phosphorous in the backbone through a ring nitrogen atom.

In all embodiments, it is absolutely critical that the hydrogel/expandable material operates under sufficient low pressure so that it does not push the stent away from the wall or alter the device configuration.

In summary, the expandable material is contained within a material, such as a semi-permeable or impermeable material so that it is retained at the site where it is needed to seal a leak. The material is selected based on the means for activation. If the material is expanded by mechanical shear or exposure to a foaming agent, these materials are provided internally within the seal, allowing an external activating agent such as an activation wire to disrupt the means for isolating the activation agent from the expandable material. If the material is activated by contact with fluid, no additional means for isolation are required if the device is stored dry prior to use, since it will activate in situ when exposed to body fluids. If the material is stored wet prior to use, a second impermeable membrane is required to keep the expandable material dry prior to activation. This will typically include a rupture site which is opened at the time of implantation to allow biological fluid to reach the expandable material through the semi-permeable material (i.e., where semi-permeable refers to a material retaining the expandable material but allowing fluid to pass). Alternatively the impermeable material may not include a rupture site but simply be removed after the device is removed from storage and washed with saline, prior to loading into the catheter, so that once the device is deployed, in situ liquid will cause the hydrogel to swell.

The properties of the different materials complement each other. For example, in the time immediately after valve deployment it is important that the material swells quickly to seal perivalvular leaks as soon as possible. Mechanical strength may be compromised in the short term to enable fast swelling. In the long term, however, it is paramount that the seal has high mechanical strength. The mechanical strength should be high enough to allow swelling and thereby “actively” conform to the gaps leading to leakage but not high enough to disturb the physical or functional integrity of the prosthesis or implant or to push the prosthesis or implant away from the wall.

A degradable material, which may be a hydrogel, that swells quickly, may be used in conjunction with a non-degradable material, which may be a hydrogel, that swells slower but has higher mechanical strength. In the short term, the degradable material capable of rapid swelling will quickly seal the perivalvular leak. Over time, this material degrades and will be replaced by the material exhibiting slower swelling and higher mechanical strength. Eventually, the seal will be composed of the slower swelling non-degradable material. It is also possible to use only one material in the seal, but in two or more different forms. For example, two different crystal sizes of hydrogels may be used in the seal, because different particle sizes of hydrogel may exhibit different properties.

2. Foams and Sponges

Alternatively, a foam generated in situ can also be used as a swellable material to form a seal. For example, a suitable matrix, such as a biocompatible polymer or crosslinkable prepolymer, may be blended with one or more foaming agents. Foaming agents include compounds or mixtures of compounds which generate a gas in response to a stimulus. When dispersed within a matrix and exposed to a stimulus, the foaming agents evolve a gas, causing the matrix to expand as fine gas bubbles become dispersed within the matrix. Examples of suitable foaming agents include compounds which evolve a gas when hydrated with biological fluids, such as mixture of a physiologically acceptable acid (e.g., citric acid or acetic acid) and a physiologically acceptable base (e.g., sodium bicarbonate or calcium carbonate). Other suitable foaming agents are known in the art, and include dry particles containing pressurized gas, such as sugar particles containing carbon dioxide (see, U.S. Pat. No. 3,012,893) or other physiologically acceptable gases (e.g., nitrogen or argon), and pharmacologically acceptable peroxides.

Other examples include changing the morphology of known hydrogel materials in order to decrease swelling times. Means for changing the morphology include increasing the porosity of the material, for example, by freeze-drying or porogen techniques. For example, particles can be produced by spray drying by dissolving a biocompatible material such as a polymer and surfactant or lipid in an appropriate solvent, dispersing a pore forming agent as a solid or as a solution into the solution, and then spray drying the solution and the pore forming agent, to form particles. The polymer solution and pore forming agent are atomized to form a fine mist and dried by direct contact with hot carrier gases. Using spray dryers available in the art, the polymer solution and pore forming agent may be atomized at the inlet port of the spray dryer, passed through at least one drying chamber, and then collected as a powder. The temperature may be varied depending on the gas or polymer used. The temperature of the inlet and outlet ports can be controlled to produce the desired products. The size and morphology of the particles formed during spray drying is a function of the nozzle used to spray the solution and the pore forming agent, the nozzle pressure, the flow rate of the solution with the pore forming agent, the polymer used, the concentration of the polymer in solution, the type of polymer solvent, the type and the amount of pore forming agent, the temperature of spraying (both inlet and outlet temperature) and the polymer molecular weight. Generally, the higher the polymer molecular weight, the larger the particle size, assuming the polymer solution concentration is the same.

Typical process parameters for spray drying are as follows: inlet temperature=30-200° C., outlet temperature=5-100° C., and polymer flow rate=10-5,000 ml/min. Pore forming agents are included in the polymer solution in an amount of between 0.01% and 90% weight to volume of polymer solution, to increase pore formation. For example, in spray drying, a pore forming agent such as a volatile salt, for example, ammonium bicarbonate, ammonium acetate, ammonium carbonate, ammonium chloride or ammonium benzoate or other volatile salt as either a solid or as a solution in a solvent such as water can be used. The solid pore forming agent or the solution containing the pore forming agent is then emulsified with the polymer solution to create a dispersion or droplets of the pore forming agent in the polymer. This dispersion or emulsion is then spray dried to remove both the polymer solvent and the pore forming agent. After the polymer is precipitated, the hardened particles can be frozen and lyophilized to remove any pore forming agent not removed during the polymer precipitation step.

Fast swelling can be achieved by preparing small particles of dried hydrogels. The extremely short diffusion path length of microparticles makes it possible to complete swelling in a matter of minutes. Large dried hydrogels can be made to swell rapidly regardless of their size and shape by creating pores that are interconnected to each other throughout the hydrogel matrix. The interconnected pores allow for fast absorption of water by capillary force. A simple method of making porous hydrogel is to produce gas bubbles during polymerization. Completion of polymerization while the foam is still stable results in formation of superporous hydrogels. Superporous hydrogels can be synthesized in any molds, and thus, three-dimensional structure of any shape can be easily made. The size of pores produced by the gas blowing (or foaming) method is in the order of 100 mm and larger.

If any portion of a superporous hydrogel is in contact with water or an aqueous medium, water is absorbed immediately through the open channels to fill the whole space. This process makes the dried superporous hydrogels swell very quickly.

Expandable sponges or foams can also be used for sealing of surgical implantations. These sponges or foams can be cut into a strips or annular shapes and either dried down or dehydrated by other means and then be allowed to rapidly re-hydrate once the device is in place. Alternatively, such materials can be hydrated and then squeezed to reduce their volume to allow these to be attached to the surgical implement and then allowed to expand to form a seal once the surgical implement is in place. Such swelling would be nearly instant.

One further benefit of sealing material in the form of sponges or foams is that their expansion can be reversible so that they can easier be retracted from their implanted position. Such sponges and foams can be made from a range of materials including, but not limited to, synthetic polymers, natural polymers or mixtures thereof. Such materials can be formed by including pore forming substances such as gas or immiscible solvents in the monomer/polymer mix prior to polymerization and/or cross-linking. By using the appropriate monomers and/or polymeric cross-linkers such sponges/foams can be made to withstand cyclic stress; such materials could also further be reinforced with compatible fibres or whiskers to increase strength and reduce the probability for breakage.

In some embodiments, these sponges or foams can be chemically attached to a base membrane or mesh used to encapsulate the sponge/foam before being fitted to the surgical device. This could be done by attaching either allylic or acrylic groups to the base substrate, either as small molecules or as long chain tentacles anchoring the expandable to the substrate preventing release of smaller particles in case of fracture.

Foams may be designed to expand without the need for the semi-permeable membrane.

C. The Support Member or Skeleton

The seal may be sufficiently flexible to conform to irregularities between the endoluminal prosthesis and a vessel wall. The band of material may include a mesh-like or a generally ring-like structure configured to receive at least a portion of an endoluminal prosthesis such that it is positioned between the portion of the prosthesis and a vessel wall. This is usually referred to as a skeleton or support member. Typically, the seal has a stent/metal backing or skeleton. The skeleton provides structure and enables crimping, loading and deployment. The skeleton can be either a balloon expanding or a self-expanding stent. The skeleton is attached to the surface of the outer membrane.

When the support member is in the second reduced radial configuration, it may form a substantially helical configuration. The helical structure of the support member provides an internal passage therein to receive at least a portion of an endoluminal prosthesis. The support member may include steel such as MP35N, SS316LVM, or L605, a shape memory material or a plastically expandable material. The shape memory material may include one or more shape memory alloys. In this embodiment, movement of the shape memory material in a pre-determined manner causes the support member to move from the first reduced radial configuration to the second increased radial configuration. The shape memory material may include Nickel-Titanium alloy (Nitinol). Alternatively, the shape memory material may include alloys of any one of the following combinations of metals: copper-zinc-aluminium, copper-aluminium-nickel, copper-aluminium-nickel, iron-manganese-silicon-chromium-manganese, copper-zirconium, titanium-palladium-nickel, nickel-titanium-copper, gold-cadmium, iron-zinc-copper-aluminium, titanium-niobium-aluminium, uranium-niobium, hafnium-titanium-nickel, iron-manganese-silicon, nickel-iron-zinc-aluminium, copper-aluminium-iron, titanium-niobium, zirconium-copper-zinc, and nickel-zirconium-titanium.

At least part of the support member may also include any one of the following combinations of metals: Ag—Cd 44/49 at. % Cd; Au—Cd 46.5/50 at. % Cd; Cu—Al—Ni 14/14.5 wt. % Al and 3/4.5 wt. % Ni, Cu—Sn App. 15 at. % Sn, Cu—Zn 38.5/41.5 wt. % Zn, Cu—Zn—X (X═Si, Al, Sn), Fe—Pt approximately 25 at % Pt, Mn—Cu 5/35 at. % Cu, Pt alloys, Co—Ni—Al, Co—Ni—Ga, Ni—Fe—Ga, Ti—Pd in various concentrations, Ni—Ti (approximately 55% Ni). The shape memory material of the support member may act as a spine along the length of the support member.

The plastically-expandable or balloon-expandable materials may include stainless steel (316L, 316LVM, etc.), Elgiloy, titanium alloys, platinum-iridium alloys, cobalt chromium alloys (MP35N, L605, etc.), tantalum alloys, niobium alloys and other stent materials.

The support member may be composed of a biocompatible polymer such as polyether or polyester, polyurethanes or polyvinyl alcohol. The material may further include a natural polymer such as cellulose ranging from low to high density, having small, large, or twin pore sizes, and having the following features: closed or open cell, flexible or semi-rigid, plain, melamine, or post-treated impregnated foams. Additional materials for the support member include polyvinyl acetal sponge, silicone sponge rubber, closed cell silicone sponges, silicone foam, and fluorosilicone sponge. Specially designed structures using vascular graft materials such as PTFE, PET and woven yarns of nylon, may also be used.

At least part of the support member may be composed of a permeable material. Alternatively, at least part of the support member may be semi-permeable. In a further embodiment, at least part of the support member may be composed of an impermeable material.

The support member may further include semi-permeable membranes made from a number of materials. Example include polyimide, phospholipid bilayer, thin film composite membranes (TFC or TFM), cellulose ester membrane (CEM), charge mosaic membrane (CMM), bipolar membrane (BPM) or anion exchange membrane (AEM).

The support member may include at least a porous region to provide a matrix for tissue in-growth. The region may further be impregnated with an agent to promote tissue in-growth. The support member itself may be impregnated with the agent or drug. The support member may further include individual depots of agent connected to or impregnated in an outer surface thereof. In one embodiment wherein the support member includes one or more capsules, the agent may be released by rupturing of the capsule. Whether the agent is held in capsules, depots, in a coating or impregnated in the material of the support member, a number of different agents may be released from the support member. For example, in an embodiment wherein the support member includes a capsule, the capsule may include an annular compartment divided by a frangible wall to separate the compartment into two or more sub-compartments. A different agent may be held in each sub-compartment. In one embodiment, the annular compartment may be divided longitudinally with at least one inner sub-compartment and at least one outer sub-compartment. Alternatively, the capsule may be divided radially into two or more sub-compartments. The sub-compartments may be concentric relative to one another. In the embodiment wherein the capsule is segmented, the different compartments may hold different agents therein.

The support member may have hooks, barbs or similar/other fixation means to allow for improved/enhanced anchoring of the sealing device to the vasculature. In addition, the support member may serve as the “landing zone” for the device when there may be the need to position the device in a more reinforced base structure, for example, in the case of valves where there is insufficient calcification for adequate anchoring, short and angulated necks of abdominal and thoracic aortic aneurysms, etc.

In all embodiments, the support member may be connected to a graft or stent by a tethering member. The tethering member may be made of an elastomeric material. Alternatively, the tethering member may be non-elastomeric and have a relatively fixed length or an appropriately calculated one for desired activation mechanism.

D. Therapeutic, Prophylactic or Diagnostic Agents

It can be advantageous to incorporate one or more therapeutic, prophylactic or diagnostic agents (“agent”) into the device, either by loading the agent(s) into or onto the structural or sealing material. The rate of release of agent may be controlled by a number of methods including varying the following the ratio of the absorbable material to the agent, the molecular weight of the absorbable material, the composition of the agent, the composition of the absorbable polymer, the coating thickness, the number of coating layers and their relative thicknesses, the agent concentration, and/or physical or chemical binding or linking of the agents to the device or sealing material. Top coats of polymers and other materials, including absorbable polymers, may also be applied to control the rate of release.

Exemplary therapeutic agents include, but are not limited to, agents that are anti-inflammatory or immunomodulators, antiproliferative agents, agents which affect migration and extracellular matrix production, agents which affect platelet deposition or formation of thrombis, and agents that promote vascular healing and re-endothelialization. Other active agents may be incorporated. For example, in urological applications, antibiotic agents may be incorporated into the device or device coating for the prevention of infection. In gastroenterological and urological applications, active agents may be incorporated into the device or device coating for the local treatment of carcinoma.

The agent(s) released from the seal or support member may also include tissue growth promoting materials, drugs, and biologic agents, gene-delivery agents and/or gene-targeting molecules, more specifically, vascular endothelial growth factor, fibroblast growth factor, hepatocyte growth factor, connective tissue growth factor, placenta-derived growth factor, angiopoietin-1 or granulocyte-macrophage colony-stimulating factor.

It may also be advantageous to incorporate in or on the device a contrast agent, radiopaque markers, or other additives to allow the device to be imaged in vivo for tracking, positioning, and other purposes. Such additives could be added to the absorbable composition used to make the device or device coating, or absorbed into, melted onto, or sprayed onto the surface of part or all of the device. Preferred additives for this purpose include silver, iodine and iodine labeled compounds, barium sulfate, gadolinium oxide, bismuth derivatives, zirconium dioxide, cadmium, tungsten, gold tantalum, bismuth, platinum, iridium, and rhodium. These additives may be, but are not limited to, mircro- or nano-sized particles or nano particles. Radio-opacity may be determined by fluoroscopy or by x-ray analysis.

In some embodiments, one or more low molecular weight drug such as an anti-inflammatory drug are covalently attached to the hydrogel forming polymer. In these cases, the low molecular weight drug such as an anti-inflammatory drug is attached to the hydrogel forming polymer via a linking moiety that is designed to be cleaved in vivo. The linking moiety can be designed to be cleaved hydrolytically, enzymatically, or combinations thereof, so as to provide for the sustained release of the low molecular weight drug in vivo. Both the composition of the linking moiety and its point of attachment to the drug are selected so that cleavage of the linking moiety releases either a drug such as an anti-inflammatory agent, or a suitable prodrug thereof. The composition of the linking moiety can also be selected in view of the desired release rate of the drug.

Linking moieties generally include one or more organic functional groups. Examples of suitable organic functional groups include secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety can be chosen in view of the desired release rate of the anti-inflammatory agents. In addition, the one or more organic functional groups can be chosen to facilitate the covalent attachment of the anti-inflammatory agents to the hydrogel forming polymer. In preferred embodiments, the linking moiety contains one or more ester linkages which can be cleaved by simple hydrolysis in vivo to release the anti-inflammatory agents.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the drug in vivo. In embodiments where the linking moiety includes a spacer group, one or more organic functional groups will generally be used to connect the spacer group to both the drug and the hydrogel forming polymer.

In certain embodiments, the one or more drugs are covalently attached to the hydrogel forming polymer via a linking moiety which contains an alkyl group, an ester group, and a hydrazide group. By way of exemplification, FIG. 1 illustrates conjugation of the anti-inflammatory agent dexamethasone to alginate via a linking moiety containing an alkyl group, an ester group connecting the alkyl group to the anti-inflammatory agent, and a hydrazide group connecting the alkyl group to carboxylic acid groups located on the alginate. In this embodiment, hydrolysis of the ester group in vivo releases dexamethasone at a low dose over an extended period of time.

Reactions and strategies useful for the covalent attachment of drugs to hydrogel forming polymers are known in the art. See, for example, March, “Advanced Organic Chemistry,” 5^{th} Edition, 2001, Wiley-Interscience Publication, New York) and Hermanson, “Bioconjugate Techniques,” 1996, Elsevier Academic Press, U.S.A. Appropriate methods for the covalent attachment of a given drug can be selected in view of the linking moiety desired, as well as the structure of the anti-inflammatory agents and hydrogel forming polymers as a whole as it relates to compatibility of functional groups, protecting group strategies, and the presence of labile bonds.

The seal can further serve as a porous matrix for tissue in-growth and can aid in promoting tissue in-growth, for example, by adding growth factors, etc. This should improve the long-term fixation of the endoluminal prosthesis. For example, the seal can be impregnated with activators (e.g., adhesive activator) that induce rapid activation of the agent (e.g., a tissue adhesive) after the agent has been released from the capsule. In other embodiments, however, the seal can be composed of different materials and/or include different features.

The agent(s) in the capsule can include adhesive materials, tissue growth promoting materials, sealing materials, drugs, biologic agents, gene-delivery agents, and/or gene-targeting molecules. In another embodiment, the one or more agent may be sheathed for delivery to a target site. Once positioned at the target site, the one or more agent may be unsheathed to enable release to the surrounding environment. This embodiment may have particular application for solid or semi-solid state agents.

Adhesives that may be used to aid in securing the seal to the lumen, or to the device to be implanted include one or more of the following cyanoacrylates (including 2-octyl cyanoacrylate, n-butyl cyanoacrylate, iso-butyl-cyanoacrylate and methyl-2- and ethyl-2-cyanoacrylate), albumin based sealants, fibrin glues, resorcinol-formaldehyde glues (e.g., gelatin-resorcinol-formaldehyde), ultraviolet-(UV) light-curable glues (e.g., styrene-derivatized (styrenated) gelatin, poly(ethylene glycol) diacrylate (PEGDA), carboxylated camphorquinone in phosphate-buffered saline (PBS), hydrogel sealants-eosin based primer consisting of a copolymer of polyethylene glycol with acrylate end caps and a sealant consisting of polyethylene glycol and polylactic acid, collagen-based glues and polymethylmethacrylate.

The hydrogel strip can be placed directly into a capsule; cast directly onto capsule material during assembly, applied using a thin film coating process such as vacuum deposition or sputter coating, by chemical bonding to the capsule material, or by electrostatic bonding to the capsule material.

Teardrop Capsule Embodiment

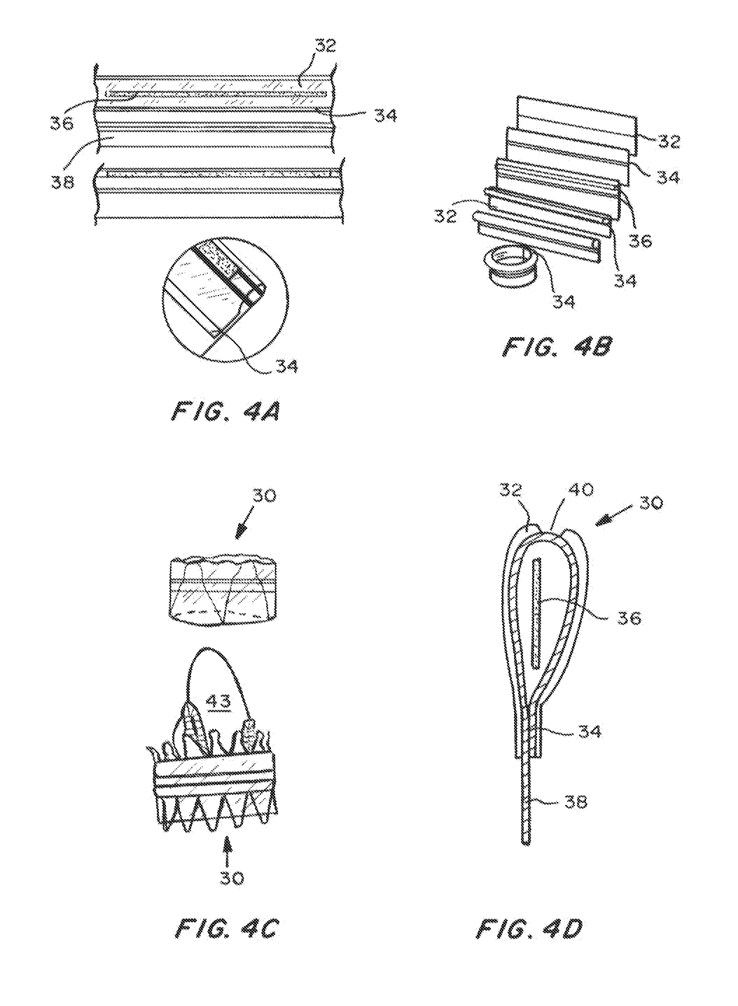

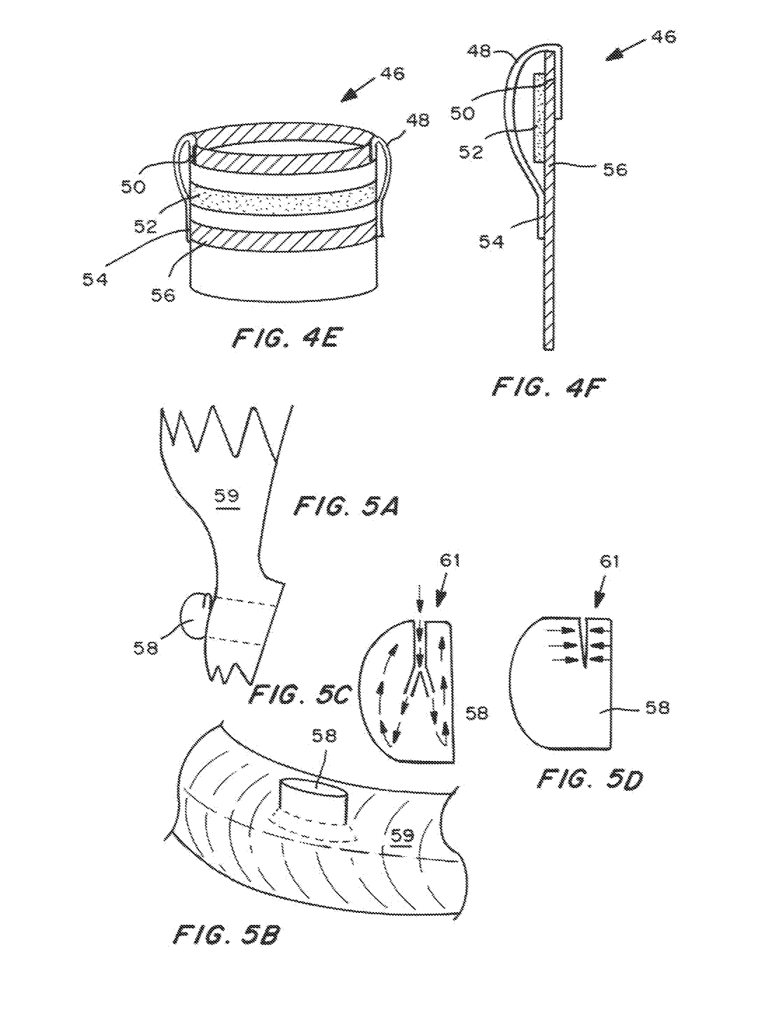

FIGS. 4A-4D are schematics of a teardrop capsule 30 which opens during diastole when the valve is closed. This is a variation of the seal capsules shown in FIG. 3 that is manufactured using straight sheets. After the assembly of the various components, the sheets are formed into a circular form in the final step to fit onto an endovascular prosthesis.

FIG. 4A is a perspective view showing the film 32 made of a polymeric material such as polyetheretherketone (PEEK), polyethylene terephthalate (PET) or polyurethane (PU); heat or laser welded seal 34; hydrogel strip 36; and mesh 38. FIG. 4B is a perspective view of the assembly of the film 32, hydrogel strip 36 and seal 34; FIG. 4C is a perspective view showing the film 32 positioned on the exterior of an expanded TAV 42; and FIG. 4D is a cross-sectional view showing the opening slit 40 from the top to allow for hydration of the hydrogel strip 36 during diastole, when the valve is closed.

This variation incorporates the following features:

The first layer is composed of a mesh 38 with the predefined porosity (approximately 50 microns) and total thickness (approximately 55 microns)

The second layer is composed of a film 32 with a predefined thickness (approximately 6 microns)

The expandable polymer (EP) 36 is encapsulated/contained between the first 38 and the second 32 layers.

The first 38 and the second 32 layers are joined by means of heat sealing processes such as laser welding, heat sealing, etc.

Alternatively, the seal 30 can be made with four layers as shown in FIG. 4D where the seal 36 is encapsulated within the mesh layers 38 and the film 32 further encapsulates the mesh layers 38. In this case the film layer 32 must contain a “slit” 40 that runs across the top layer of the film.

FIGS. 4E and 4F are cross-sectional views of the teardrop capsule 30 of FIGS. 4A-4D, manufactured a different way. The seal 46 is manufactured directly into the circular (or appropriate closed shape) by using specific jigs and fixtures to perform the joining/welding operations. This eliminates one extra step in manufacturing, i.e., the last step of making a linear profile into a circular of FIGS. 4A-4D.

FIGS. 4E and 4F shows the film 32 overlaying the mesh 38, having the hydrogel strip 36 positioned thereon, overlaid by the sealed film 32. The D profile capsule 46 opens during systole, when the valve is open, showing the blow molded D balloon formed by the film 48 sealed over the hydrogel strip 52 positioned on the mesh 56, and the assembly of the TAV device with seal. The exposed mesh 56 allows for hydration of the hydrogel strip 52 during systole, while maintaining a much lower profile assembly given reduced layers of material across any section.

Ice Bag Filling Seal

As shown in FIGS. 5A-5D, a seal 58 including a valve opening 61, which closes as the seal 58 fills with liquid, can be used to expand the seal in situ. This seal 58 uses positive pressure to fill with blood. There is no hydrogel in this embodiment.

This is an ultralow profile seal system that essentially consists of an annular bag 59 made from film. The annular film bag 59 further consists of one or more one-way valves 61 designed such that the valve 61 will open by virtue of the pressure of the blood within the vasculature and allow the blood to flow into the bag and fill it (FIG. 5C). Once the bag 58 is full the one-way valve 61 will close by virtue of internal pressure of the blood within the bag 58 (FIG. 5D). This system can further contain a means to activate the functioning of the valve (i.e. expose the orifice to the blood) once the endovascular prosthesis is deployed within the vasculature, allowing on-demand activation of the seal.

D Profile Capsule

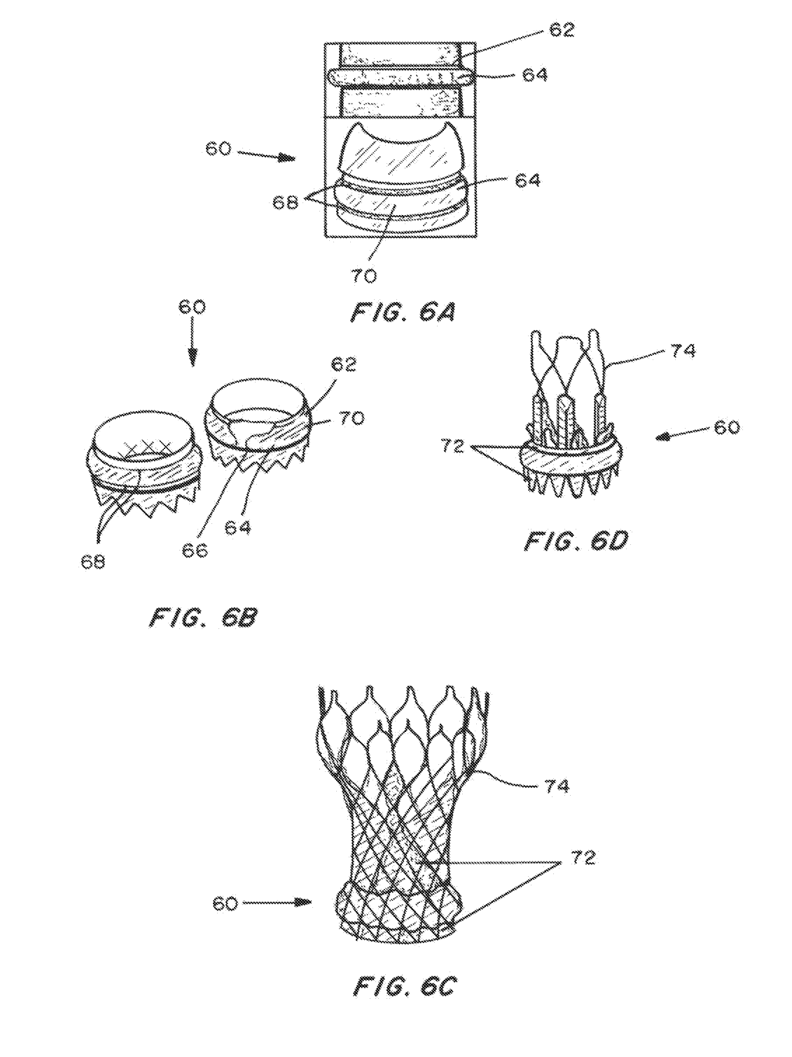

FIGS. 6A-6D are views of a D profile capsule 60. The film 62, formed of a material such as PEEK, PET, or PU, is blow molded to form a “D” balloon 64 over a mesh 66. The seam 68 is heat or laser welded to seal a hydrogel strip 70 between the film 62 and the mesh 66. This capsule assembly 60 is then sutured to the tissue skirt assembly 72 of the TAV device 74.

This is a variation of the sealable capsules shown in FIG. 3 that has a specific cross sectional profile in the shape of the letter “D”. The flat portion of the D profile lies in abutment to the prosthesis while the curved portion of the D profile lies in abutment to the anatomy/blood vessel. The flat and the curved portions can be manufactured/managed in the same manner as outlined in FIGS. 4A-4F by using mesh, film or a combination thereof.

The specific D profile is obtained by the process of blow molding when it is made from a film or by a process of 3D weaving when it is made from a mesh.

The functional advantage of the D profile is that once the seal 60 is activated and the hydrogel 70 swells, the seal 60 will only swell towards the curved section of the D profile and will have no swelling/deformation of the flat portion of the D profile. This in turn ensures that the prosthesis is not pushed inwards by virtue of the expansion of the seal.

E. Devices for Placement of Devices with Sealing Means

In a preferred embodiment, the sealing means is positioned posterior to the prosthetic implant, and is expanded or pulled up into a position adjacent to the implant at the time of sealing. This is achieved using sutures or elastic means to pull the seal up and around the implant at the time of placement, having a seal that expands up around implant, and/or crimping the seal so that it moves up around implant when implant comes out of introducer sheath. This is extremely important with large diameter implants such as aortic valves, which are already at risk of damage to the blood vessel walls during transport.

A key feature of the latter embodiment of the seal technology is that it enables preservation of the crimped profile of the endoluminal prosthesis. The seal technology is crimped distal or proximal to the prosthesis. In one aspect of this technology, the seal is aligned with the prosthesis by expansion of the seal. In another aspect, the seal zone of the prosthesis is aligned with the seal zone prior to expansion of the prosthesis by use of activation members. In yet another embodiment, the seal is aligned with the seal zone of the prosthesis prior to prosthesis expansion by use of activation members, which can be made of an elastic or non-elastic material.

In a further embodiment, the endoluminal device may further include one or more engagement members. The one or more engagement members may include staples, hooks or other means to engage with a vessel wall, thus securing the device thereto.

A stent-balloon-TAV-capsule has been developed with a very low profile. The capsule is delivered within the TAV using a stent. The capsule is flipped out and over the bottom edge of the TAV immediately prior to positioning. It is important to center the valve within the stent or it will not flip over correctly.

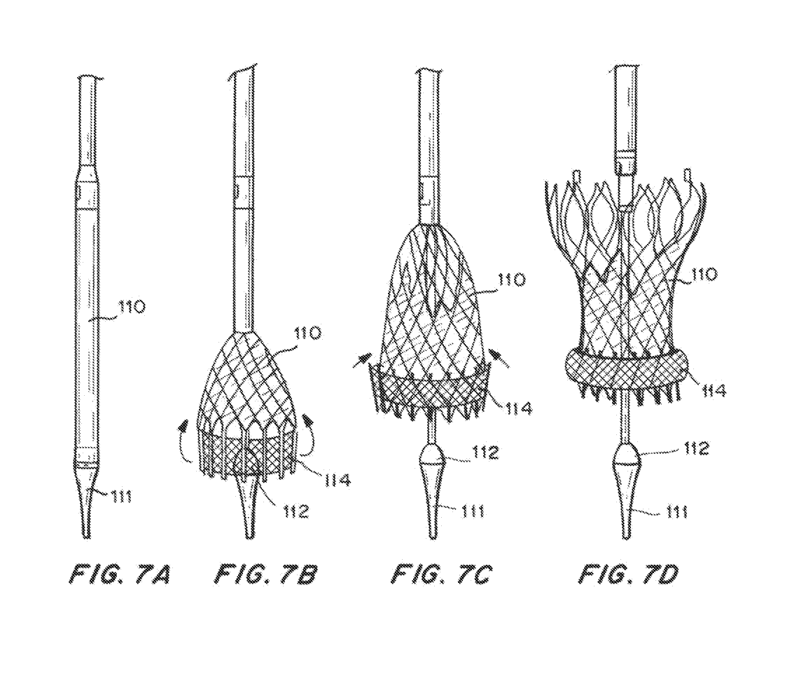

FIGS. 7A-7D are perspective views of the TAV 110 with the stent 111 (FIG. 7A), the TAV 110 expanded and the capsule 114 ready to flip over (FIG. 7B), the TAV 110 expanded and pulled back with the capsule 114 flipped over (FIG. 7C), and the TAV 110 and flipped over capsule 114 expanded (FIG. 7D). FIGS. 7A-7D:

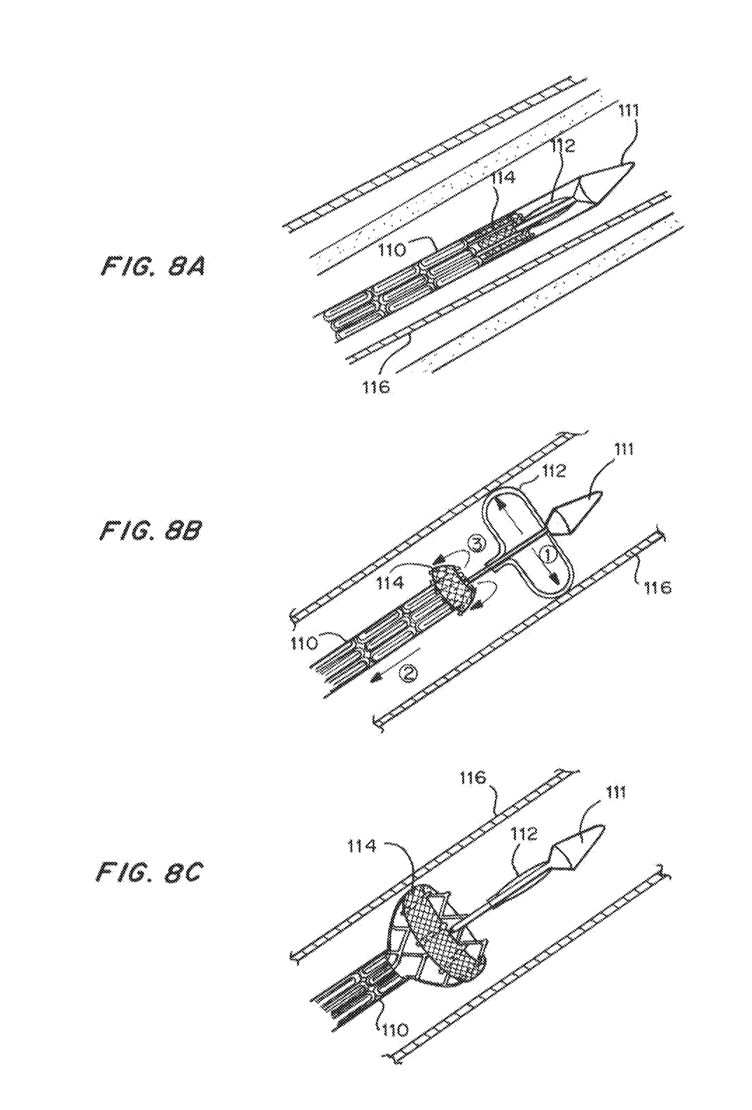

FIG. 8A shows a TAVI stent 110/111 with a flippable HG capsule 114 in a catheter 116. The balloon 112 expands to center the TAV, as shown in FIG. 8B, and the capsule 114 flips over the outside of the TAVI stent 110 when the catheter 116 is pulled back; showing the balloon inflation centering the catheter 116. FIG. 8C shows the capsule 114 flipped over the TAV 110. This is a further development of the “flippable strap” concept in which the balloon 112 needs to be incorporated in the catheter 116 within which the device with the “flippable strap” is loaded for delivery. The balloon 112 has to be positioned in front of the device 110. The balloon 112 is essential to allow for centering of the device 110 within the catheter 116 when the “flippable strap” 114 flips. This is done by inflating the balloon 112 (FIG. 8B) and then deflating it once the flipping procedure is complete (FIG. 8C).

F. Additional Encapsulation of Sealing Means for Increased Shelf-Life

The seal may be sterile packaged for distribution and use. In the alternative, it may be packaged as part of, or in a kit with, the device it is designed to seal, such as a TAV or stent. This additional encapsulation prevents the activation of the expandable material during storage within solutions (e.g. glutaraldehyde, alcohol) by acting as a 100% moisture barrier.

Heart valves, both transcatheter and surgical, are stored in glutaraldehyde or similar solutions primarily to preserve the tissue component of the device. Before the device is implanted, it is prepared for implantation by removing it from the solution and rinsing it thoroughly so that all the glutaraldehyde is washed off.

Although the outer impermeable layer of the sealing device/capsule is meant to prevent any penetration of water from the glutaraldehyde into the capsule, there is a likelihood that the thickness may be insufficient given the profile constraints and as a result there may only be a limited shelf-life that may be obtained. In order to obtain an increased shelf-life where the encapsulated expandable material remains in its desirable unexpanded state until introduced within the body, an additional impermeable layer may be needed. This additional impermeable layer is not required once the device is removed out of the storage solution, and is rinsed to wash all the glutaraldehyde away. This will typically be removed after removing the device from the storage fluid and just before implantation.

To make the sealing means low profile, the thickness of the outer and inner membranes has to be kept to the minimum. If the sealing device is stored submerged in a solution, as in the case with transcatheter valves, for its shelf-life, the low profile, thin membranes may allow moisture to permeate through them and thereby risk the premature activation of the sealing means. Therefore, an additional means is necessary to ensure the appropriate shelf-life of the sealing device can be obtained.

This additional encapsulation layer is removable and is designed to have a mechanism which enables easy peeling of the hermetic sealing capsule/layer so that this layer can be removed just before loading and crimping of the prosthesis into the delivery catheter, before it is delivered into the vasculature. The layer can be removed using different means, including peeling off, cracking off, melting off, vapouring off after the rinsing process is complete and the device is ready to load.

The additional encapsulation layer may be designed with a mechanism so that it can be attached to the device assembly with the sealing means during the assembly process by suturing or other appropriate means such that the removal process insures that integrity of the sealing means and its assembly with the base device remains completely intact.

A moisture impermeable film composite includes a combination of polymer films, metalized polymer films and metal films. The polymer layers can be formed of polyether ether ketone (PEEK), polyethylene terephthalate (PET), polypropylene (PP), polyamide (PI), polyetherimide (PEI) or polytetrafluoroethylene (PTFE), or other similar materials. Polymer films may or may not be mineral filled with either glass or carbon. Polymer films will have a thickness of 6 μm or above. Metal films and coatings include aluminum, stainless steel, gold, mineral filled (glass and carbon) and titanium with a thickness of 9 μm or above. Coatings can be applied with processes such as plasma vapor deposition, press lamination, vacuum deposition, and co-extrusion. Metal films can be laminated to polymer films via press lamination.

O-Ring Sealing Package

The hydrogel strip is very thin, less than one mm in thickness. Typically it must be further sealed in a metal foil laminate to keep the hydrogel strip from hydrating, since all polymers eventually allow permeation of fluid. A metal-polymer laminate has been developed as a means to allow the seal to be stored in a liquid environment, since the valve is stored in an immersed state within a solution such as glutaraldehyde. Just using an impermeable membrane may not be sufficient if the membrane is too thin or if there is fluid permeating through the material over time. It may not be possible to make the membrane sufficiently thick or impermeable to prevent fluid passage over time. This will adversely affect shelf life as any leakage of fluid will cause the seal to swell.

The removable casing is made of sheet metal or thick plastic/polymer, and has the following features:

It is in a “U” shape that allows for complete insertion of the seal within the “U” cavity.

The open end of the “U” cavity has O-rings and a locking mechanism that when activated, for example, using a snap-fit mechanism, compresses the O-rings to bring them under pressure, thereby allowing the formation of an air-tight seal.

This in turn prevents any fluid from entering into the “U” cavity where the seal is housed.

Before loading of the device in the catheter, the locking mechanism is deactivated.

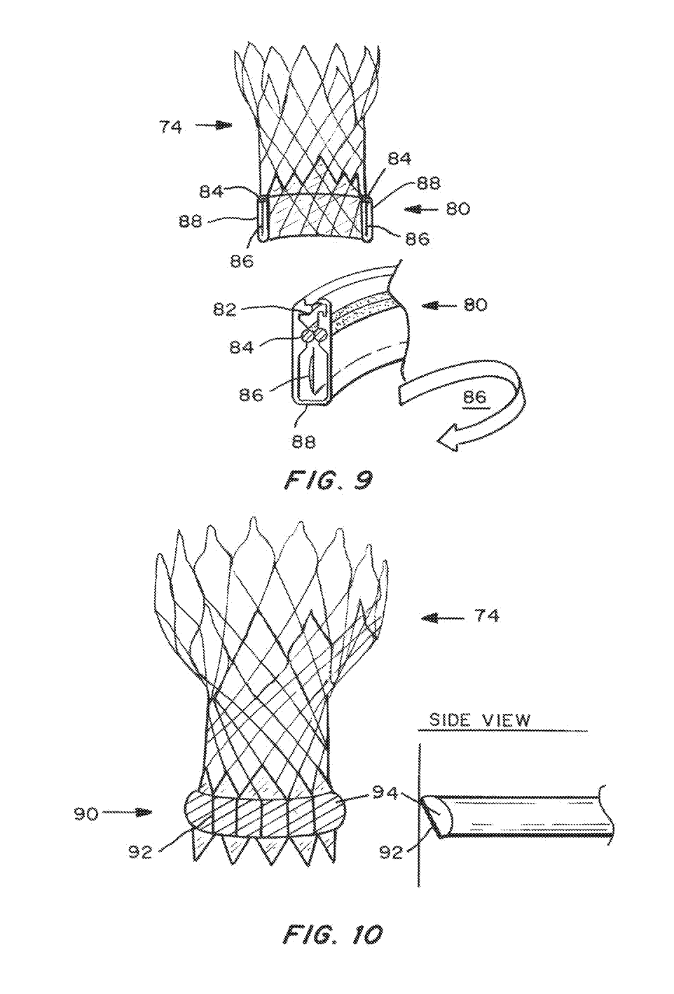

FIGS. 9A-9B are views of an O-ring casing 80, showing a U shaped casing 88 that encapsulates the seal assembly 86 during storage, preventing hydration of hydrogel by preservative, such as glutaraldehyde. The U shaped casing 88 encompasses and excludes liquid from the seal capsule assembly 86. The U shaped casing 88 is snapped together at two interlocking pieces of the snap fit assembly 82, and fluidically sealed by two O-rings 84.

Foam Seal

A different device was developed for the seal when the swellable material is a foam instead of a hydrogel. The hydrogel by virtue of its polymerization characteristics has a tendency to exert a “swelling force” as it polymerizes/swells. This is not present with a foam, as the foaming action happens ex vivo. The “swelling force” for the hydrogel allows for the conformation of the seal to expand into the “gaps” to fill any leak sites. The foam cannot do this by itself, and therefore the seak must be supported by spring struts which help push the foam into the “gaps”. The spring struts are made from Nitinol material, and are activated once the device is removed from the catheter and deployed within the body.

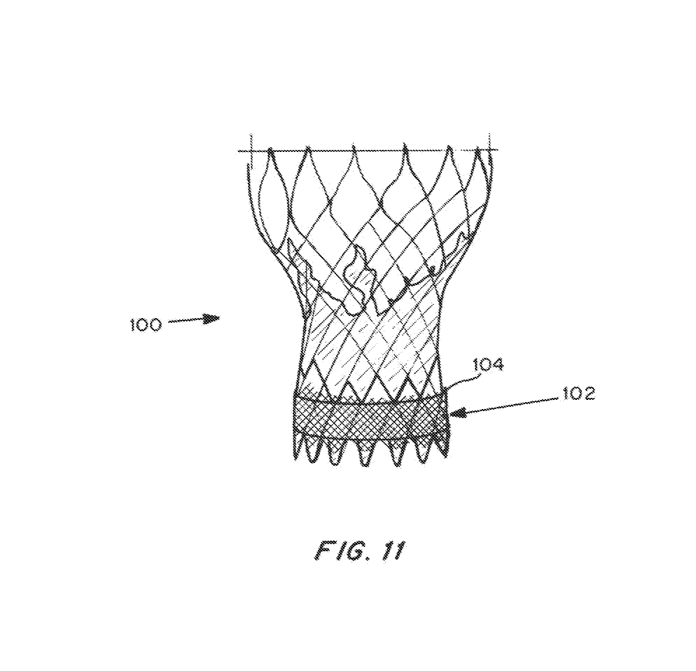

FIGS. 10A and 10B are views of a foam seal 90 which is attached to the inside of TAV struts 94 so that the foam 90 is forced through the struts 94 and into leak sites using integrated spring struts 92 or using a balloon.

Dissolvable Film

Another variation of the seal incorporates an impermeable membrane or film that is “dissolvable” under specific conditions, such as a temperature, pH or a combination thereof. The “dissolvable” impermeable layer remains intact in the storage fluid (glutaraldehyde), but once the device is introduced into the vasculature, it will dissolve exposing the permeable layer and therefore the EP within.

FIG. 11 is a view of a TAV 100 with a dissolvable film 102 to seal the seal capsule 104 to prevent hydration. The dissolvable film is made of a material such as polyvinyl alcohol or EUDRAGIT® (polyacrylamide) which dissolves at physiological pH, in isotonic fluid, or in a specific liquid.

Solid Silicone Core

In another embodiment to prolong shelf-life of the seal when stored in a environment that could be contaminated by the liquid used to store the valve, which is stored in an immersed state within a solution like glutaraldehyde. The potential limitation of the device shown in FIG. 9 is that, given the “U” profile of the cavity, the section of the seal that is within the “U” and next to the curved portion of the “U” cannot be attached to the prosthesis as it rests within the “U” cavity. There may be occasions where complete attachment of the seal to the prosthesis is a requirement for the functionality of the device.

Accordingly, rather than placing the seal within a fluid tight container, a compliant “plug” is inserted within the stent. This plug is made of the same materials as the O-ring (rubber, silicone, etc.) of the device of FIG. 9. Around the outside of the stent a sleeve made of the same or similar compliant material as the plug is placed such that the seal is sandwiched between the outer sleeve and the inner plug. The sleeve is compressed against the inner plug by means of applying a mechanical pressure, for example, by using a ratchet mechanism belt or other oversized compliant material belts. These belts can be attached to either top or the bottom end or both ends of the sleeve. As a result of this structure the end result will be the same as that obtained by the O-rings in FIG. 9, except that in this case both the top and bottom ends of the SEAL are secured.

Both the sleeve and the plug can be designed to have a pre-determined shape in order to accommodate to the shape and design of the stent/prosthesis i.e. appropriate grooves, etc. can be cut into the sleeve and the plug to ensure an fluid-tight contact can be made possible between the two. During the deployment procedure, just before the device is loaded, the belt or belts can be removed that will lead to the relief of pressure between the sleeve and the plug so that the two can now be separated and removed easily further allowing for the removal of the “impermeable barrier” and crimping/loading of the prosthesis within the catheter.

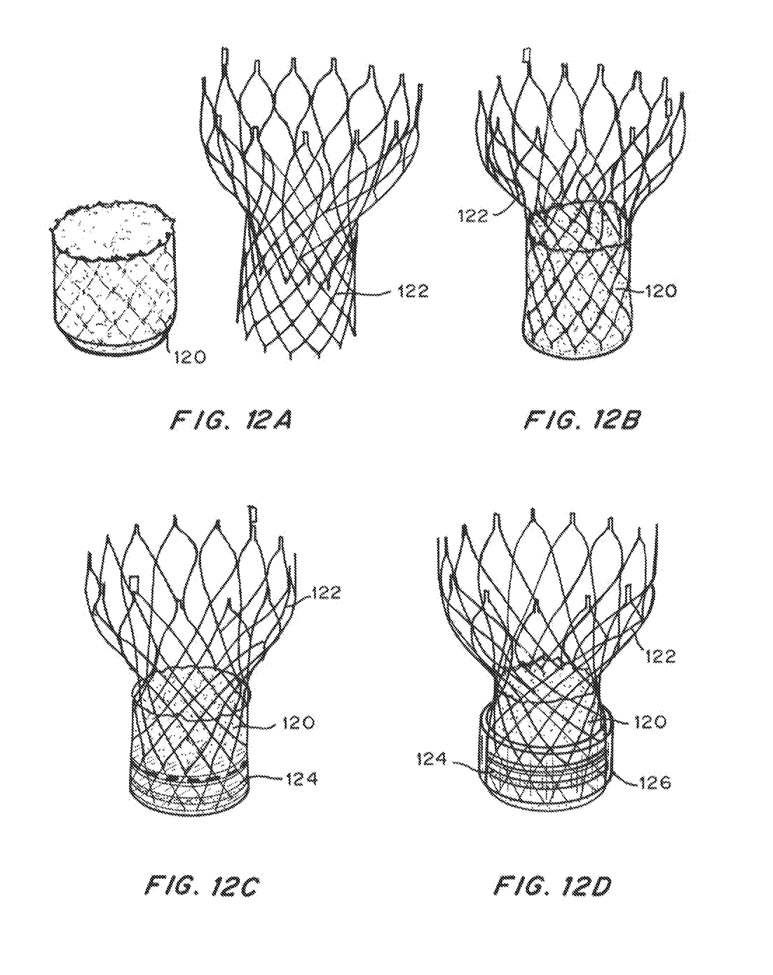

FIGS. 12A-12E are perspective views of a pre-cut, molded solid silicone core 120 (FIG. 12A) that sits inside of the valve 122 (FIG. 12B) with the metal struts of the TAV 122 sitting flush within recesses of the silicone core 120 (FIG. 12C), wherein the seal capsule 124 is on the outside or inside of the TAV frame 122 (FIG. 12D), with the maximum height of the silicone core 120 to allow for suturing on the upper portion of the silicone core 120 to the TAV 122. A silicon sleeve 126 is placed over the TAV frame 120 and capsule 124 assembly, sandwiching the stent frame and capsule by virtue of the elastic properties of the band and mechanical pressure from the ratchet mechanism (FIG. 12E).

Metal Laminate

In this embodiment of the seal shown in FIG. 3, the impermeable layer is designed using metallic film or a metallic film with a polymer laminate. This metallic film acts as a barrier during the storage of the device in glutaraldehyde, and is designed to be “peeled off” once it is removed and just before loading of the device within the catheter. This metallic barrier film can be in addition to the impermeable film as shown in FIGS. 4A-4D.

The main features include:

Pull tabs:

Horizontal pull tab for “peeling off” the metallic barrier film along the score line. This “peeling off” action breaks the watertight seal/barrier.

Vertical pull tabs that allow for the seamless removal of the remaining metallic barrier film once that horizontal pull tab is removed.

A premade score line that allows for a clean “peeling off” mechanism.

The design includes heat sealing the different components together in such a manner that the metallic barrier film can be removed cleanly in two parts.

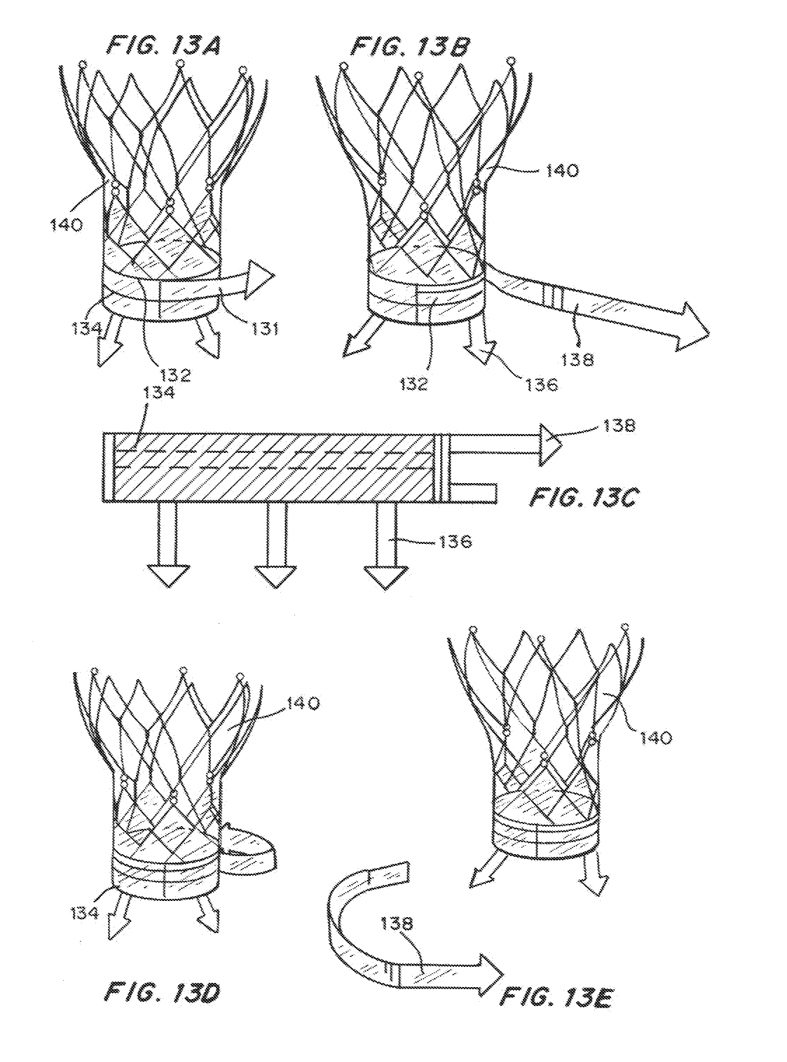

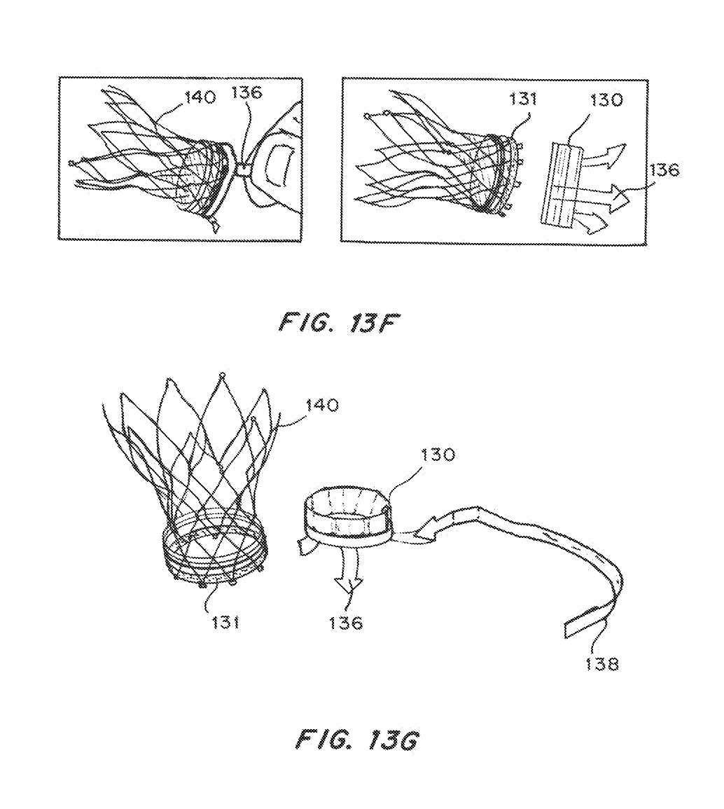

This is an additional detail within FIG. 13 that shows a cross section view of the metallic barrier film used. It is shown that in this case there is a polymer layer (low density PE) that is laminated on the inner side of the metal. Such lamination helps with achieving a “weld” through the mesh as the polymer melts and flows from between the pores of the mesh to finally solidify and form one unit.

Such a structure allows for getting a seal through a mesh, allows for clean removal of the barrier layer during the “peeling off” process and allows the mesh to remain completely intact.

FIGS. 13A-13E are perspective views of the Metronic TAV 140 with a metal polymer laminate 130 surrounding the capsule 131, heat sealed in front and back (FIG. 13A), with the tab 138 pulled around the stent frame 140 breaking the heat seal bond 132 and the bottom pull tabs 136 pulled to remove the protective cover to prevent hydration of the capsule 131 during storage (FIG. 13B), shown in cross-section in FIG. 13C, and completely removed as shown in FIGS. 13D and 13E. FIGS. 13F-13G show the TAV 140 of FIGS. 13A-13E, with the remainder of the metal-polymer film 138 pulled away from the capsule 131 via the bottom pull tab 136 (FIG. 13E), detaching the protective covering 130 completely (FIG. 13F), leaving the sealed TAV 140 separate from the covering 130 (FIG. 13G). The metal laminate includes an outer metal foil layer, weakened score path to peel and break the heat seal bond, and an inner polymer layer, formed of a polymer such as low density polyethylene (ldpe), heat sealed through the mesh to bond with the polymer (ldpe) on the inside of the device.

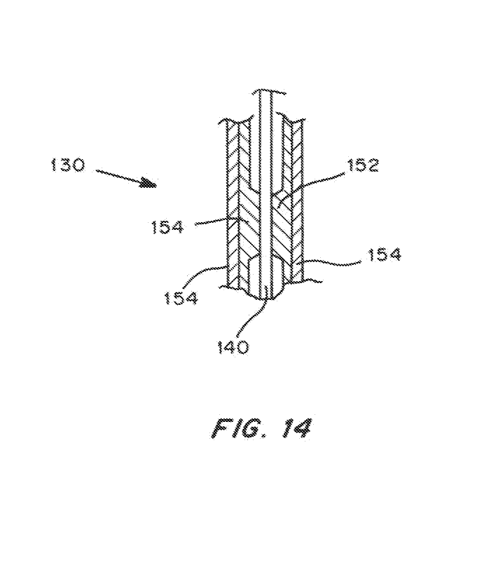

FIG. 14 is a cross-sectional view of the metal laminate 130, showing the polymer 154 melting through the mesh of the TAV 140, and the outer metal layer 154.

G. Packaging for Expandable Seal Devices

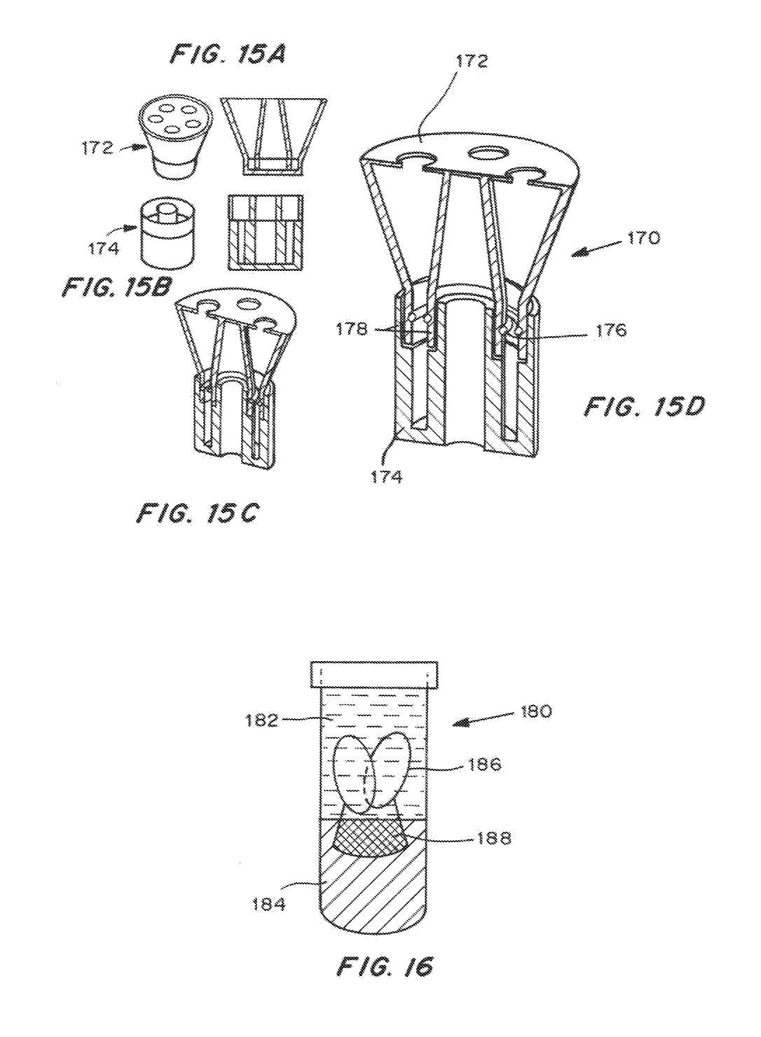

FIGS. 15A-15Dd show a packaging case 170, having an upper compartment 172 and a lower compartment 174. The upper and lower parts are screwed together at 178, and sealed using O-rings 176.

This is a completely different approach to maintain the shelf life of the seal when the device with the seal is stored in an immersed state within a solution like glutaraldehyde. This approach entails redesigning the storage container instead of modifying the seal. By doing so, many of the manufacturing hurdles related to the seal can be avoided, thereby making it easy to manufacture and less risky to handle during preparation, crimping and loading into the catheter before the procedure.

In this embodiment, the container is designed in two parts, a top part designed to house the stent and a bottom part designed to house the seal. The top and the bottom parts are attached together by means of a screw mechanism such that two O-rings at the interface compress against each other, thereby shielding the seal portion of the container from any fluid contact. The top portion of the container contains a fluid such as glutaraldehyde, thereby keeping the tissue leaflets hydrated and preserved, while keeping the seal in the bottom portion dry. The shapes of the top and bottom portion of the containers can be changed to accommodate to the design/shape of the device under consideration.

FIG. 16 shows packaging 180 for the TAV 186 with silicone core 188 and ratchet band shown in FIGS. 12A-12D, which is placed into a container 184 of a liquid silicone. The silicone solidifies to seal the capsule. The TAV and stent 186 is released when the packaging 180 is opened

This is a means to achieve shelf life when the TAV device with the seal 186 is stored in an immersed state within a solution like glutaraldehyde. This approach entails a step-by-step isolation procedure for the seal once it has been assembled onto the device. This approach does not need any modification to the seal with extra impermeable layers, or any significant modifications to the shape of the container. The steps for achieving the isolation are:

A silicone (or similar compliant material) plug 188 is inserted on the inner side of the device as shown in FIG. 12. This inner plug 188 covers and/or secures the inner portion of the SEAL from within the inner lumen of the device.

The device with the plug is placed within the container and the bottom of the container 184 until the height of the top section of the inner plug is filled with quick setting polymer of lesser compliance than the inner plug, such as a silicone, epoxy, etc.

The seal is now compressed between the inner plug and the outer layer of lesser compliant (or more rigid) material. The difference in compliance results in mechanical pressure that forms a water tight interface between the inner plug and the outer layer.

Once the watertight interface is made, the top (or remaining) portion of the storage container can then be filled with fluid (glutaraldehyde), thereby isolating the seal from the storage fluid.

In order to remove the device, the storage fluid can be drained off—the storage jar/container can be broken to expose the set/polymerized outer polymer. The difference in compliance allows for the easy separation of the outside polymer with the stent and further with the inner plug. The device is now ready to be loaded within the catheter.

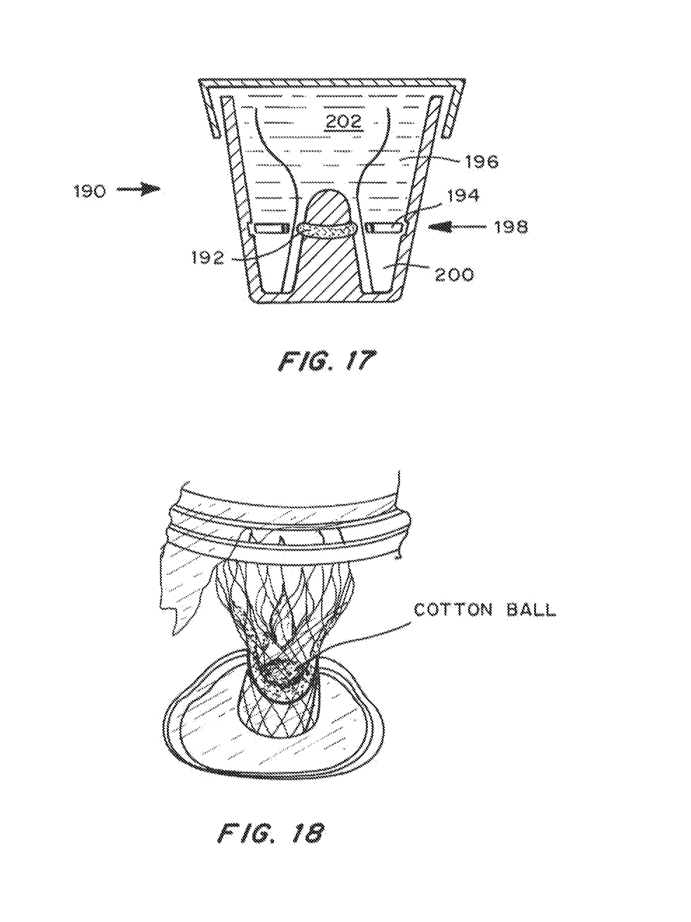

Another embodiment is shown in FIG. 17. This package 190 includes a tapered jar 198 and compression disc 194 to separate the liquid 196 around the device from the hydratable seal 200 which is located in the lower dry portion of the jar 198.

This approach is also to modify the container, rather than the seal. The container design has the following features:

Around the central core of the container there is an extruded “mountain” like protrusion around which the SEAL portion of the device sits. The height of this “mountain” section is the same as that of the inner plug as discussed in FIG. 16, with the difference being the inner plug of FIG. 16 was made of compliant material and this “mountain” is rigid. An O-ring is placed on top of the “mountain” and on the outside of the SEAL a compression disc is placed that pushes against the inner O-ring. This inner O-ring and the outer compression disc isolate the bottom portion of the device. Moreover, because the bottom portion of the device contains the seal, the seal remains secluded from the upper portion that contains the tissue leaflets of the valve and therefore has to be wet. Once the seal is isolated, the storage fluid can be poured into the top portion of the container. The bottom section below the O-ring, compression ring interface remains dry.

FIG. 18 is a diagram of another container showing an absorbant material such as a cotton ball on the tissue. In this embodiment, neither the device nor the storage container needs to be modified. Instead, the absorbant material is permeated with the storage fluid (glutaraldehyde) so that the tissue leaflets constantly remain wet. The absorbant remains in constant contact with the tissue leaflets to prevent drying, while not contacting the seal.

### III. Methods of Use

The device and seal can be utilized for sealing in a variety of tissue lumens, including cardiac chambers, cardiac appendages, cardiac walls, cardiac valves, arteries, veins, nasal passages, sinuses, trachea, bronchi, oral cavity, esophagus, small intestine, large intestine, anus, ureters, bladder, urethra, vagina, uterus, fallopian tubes, biliary tract or auditory canals. In operation, the endoluminal prosthesis is positioned intravascularly within a patient so that the prosthesis is at a desired location along a vessel wall. A balloon or other expandable member is then expanded radially from within the endoluminal prosthesis to press or force the apparatus against the vessel wall. As the balloon expands, the activation wire is triggered, rupturing the capsule and causing the seal to swell, and in some embodiment, releasing agents. In one embodiment, the agent includes an adhesive material and when the capsule ruptures, the adhesive material flows through the pores of the seal. As discussed above, the seal can control the flow of the adhesive to prevent embolization of the adhesive material.

In specific embodiments, the device may be used to seal a graft or stent within an aorta of a patient. In a further embodiment, the device may be used to seal an atrial appendage. In this embodiment, the device may deliver an agent to effect the seal of a prosthetic component across the opening to the atrial appendage.

In a further embodiment, the device may be used to seal a dissection in a vessel. In this embodiment, the support member is positioned adjacent the opening of the false lumen and an intraluminal stent subsequently delivered thereto. Upon radial expansion of the stent, the support member is caused to release adhesive therefrom to seal the tissue creating the false lumen against the true vessel wall.

In a further embodiment, the device is used to seal one or more emphysematous vessels.

In a still further embodiment, the device may be used to seal an artificial valve within a vessel or tissue structure such as the heart. An example includes the sealing of an artificial heart valve such as a TAV. It is envisaged that the seal provided by the present device will prevent paravalvular leaks.

The device with seal is inserted in a manner typical for the particular device. After reaching the implantation site, the seal is ruptured and the seal expands to seal the site. The guidewire and insertion catheter are then withdrawn and the insertion site closed.

The seal may be sterile packaged for distribution and use. In the alternative, it may be packaged as part of, or in a kit with, the device it is designed to seal, such as a TAV or stent.

The present invention will be further understood by reference to the following non-limiting examples.

### Example 1

### Preparation of Hydrogel with Rapid Swelling

Studies to identify hydrogels having substantial swelling in a short time were performed. The main factors that influence swelling of a hydrogel based on polymerisation and cross-linking of synthetic monomers are:

Type of monomer

Type of cross-linker

Concentration of monomer and cross-linker in the gel

The ratio of monomer to cross-linker

Acrylic acid polymers are capable of rapid swelling and are regarded as having good biocompatibility. A number of commercially available cross-linkers can be used to crosslink the polymers to form a hydrogel. These include Bis acrylamide, di(ethylene glycol) diacrylate, and poly(ethylene glycol) diacrylate (MW 500 Da).

Materials and Methods

Studies were conducted to identify appropriate combinations of acrylic acid concentration, type of cross-linker, concentration of cross-linker and ratio of monomer to cross-linker. The basic composition of the formulations used to make the gels is shown in Table 1. These were prepared as follows:

Mix acrylic acid with cross-linker and 50% of the necessary water, adjust pH to neutral with 15M sodium hydroxide and adjust the total volume with water.

Degas the solution under vacuum in a desiccator or other suitable container.

Add initiators (APS and TEMED), mix well and leave to gel overnight.

Test for mechanical properties and swelling.

After forming the gels in small beakers or Falcon tubes, the gels were cut into small pieces and dried until complete dryness. Small pieces of gel were then collected and re-swollen in phosphate buffered saline (PBS). The weight of the gel pieces were then recorded at regular intervals.

Results

Compositions and swelling data are shown in Tables 1 and 2.

TABLE 1
Swellable Formulations

| Gel | 2 | 3 | 5 | 6 | 21 | 29 | 25 |
|---|---|---|---|---|---|---|---|
| AA | 40 | 40 | 40 | 20 | 20 | 15 | 10 |
| BIs | 0.4 | 0.4 | 0.4 | 0.2 | 0.1 | 0.05 | 0.02 |
| APS | 0.33 | 0.08 | 0.08 | 0.08 | 0.08 | 0.08 | 0.08 |
| TEMED | 0.33 | 0.8 | 0.08 | 0.08 | 0.1 | 0.1 | 0.1 |
| STATUS | Swelled | Swelled | Swelled | Swelled | Swelled | Swelled | Swelling |

| Gel | 17 | 23 | 19 | 26 | 28 |
|---|---|---|---|---|---|
| AA | 20 | 15 | 10 | 10 | 5 |
| PEG | 0.1 | 0.05 | 0.05 | 0.02 | 0.025 |
| APS | 0.08 | 0.08 | 0.08 | 0.08 | 0.08 |
| TEMED | 0.1 | 0.1 | 0.1 | 0.1 | 0.1 |
| STATUS | Swelled | Swelled | Swelled | Swelling | Swelling |

| Gel | 18 | 24 | 27 |
|---|---|---|---|
| AA | 20 | 15 | 10 |
| DEG | 0.1 | 0.05 | 0.02 |
| APS | 0.08 | 0.08 | 0.08 |
| TEMED | 0.1 | 0.1 | 0.1 |
| STATUS | Swelled | Swelled | Swelling |

TABLE 2
Analysis of Hydrogels made with the PVA cross-linker

| Gel 23 rep 1 | | Gel 23 rep 2 | | Gel 23 rep 3 | | Gel 23A rep 1 | | Gel 23A rep2 | | Gel 23A rep 3 | |
|---|---|---|---|---|---|---|---|---|---|---|---|
| App. | rectangular | App. | triangle | App. | rectangular | App. | triangle | App. | trapezoid | App. | trapezoid |
| Shape | | Shape | | Shape | | Shape | | shape | | Shape | |
| Side 1 | 2 | Base | 2 | Side 1 | 1.5 | side 1 | 2 | base 1 | 1 | base 1 | 1.5 |
| (mm) | | (mm) | | (mm) | | .(mm) | | (mm) | | (mm) | |
| Side 2 | 2 | Height | 5 | side 2 | 1.25 | side 2 | 3 | base 2 | 1.5 | base 2 | 2 |
| (mm) | | (mm) | | (mm) | | (mm) | | (mm) | | (mm) | |
| Thickness | 0.33 | Thick- | 0.25 | thickness | 0.625 | thickness | 0.33 | height | 1 | height | 1 |
| | | ness | | (mm) | | (mm) | | (mm) | | (mm) | |
| (mm) | | (mm) | | | | | | thick- | 0.25 | thick- | 0.585 |
| | | | | | | | | ness | | ness | |
| | | | | | | | | (mm) | | (mm) | |
| Volume | 3.33333 | | 1.25 | | 1,17187 | | | | 0.3125 | | 1.02375 |
| (mm³) | | | 12.8507 | | 5 | | 1 | | 3.6545 | | 6.78654 |
| Surface | 3333 | | 8106 | | | | 8.7748 | | 0 | | 9883 |
| Area | 10.6666 | | 10.2806 | | 7.1875 | | 5 | | 8497 | | 6.62910 |
| (mm³) | 6667 | | 2485 | | 6.13333 | | 1773 | | 11.634 | | 8555 |
| SA to V | 8 | | | | 3333 | | 8.7748 | | 4 | | |
| ratio | 0.003 | | 0.003 | | 0.0009 | | 5 | | 2719 | | 0.0011 |
| Beginning | 0.00225 | | | | 0.00076 | | 1773 | | | | 0.00107 |
| Mass (g) | | | 4.93333 | | 8 | | | | 0.0008 | | 4481 |
| Density | 4.5 | | | | 8.66666 | | 0.0025 | | | | 18.6363 |
| (g/mm²) | | | 3333 | | 6467 | | 0.0025 | | 0.0025 | | 6364 |
| 5 min. | | | | | | | 9.1923 | | 6 | | |
| swell | | | | | | | 0 | | | | |
| ratio | | | | | | | 7692 | | 16.125 | | |

ALL Gel 23 SAMPLES DISSOLVED AFTER THE 3 MINUTE POINT

| Gel 23B rep 1 | | Gel 23B rep 2 | | Gel 23B rep 3 | | Gel 23C rep 1 | | Gel 23C rep2 | | Gel 23C rep 3 | |
|---|---|---|---|---|---|---|---|---|---|---|---|
| App. | triangle | App. | | App. | house | App. | square | App. | triangle | App. | rectangle |
| Shape | | Shape | | Shape | | Shape | | Shape | | Shape | |
| base | 4.5 | Base | 3 | bottom | 1.5 | side 1 | 3 | base | 3 | side 1 | 1.5 |
| (mm) | | (mm) | | (mm) | | (mm) | | (mm) | | (mm) | |
| height | 5 | Height | 0.441 | side | 2.5 | side 2 | 0.729 | height | 3 | side 2 | 2 |
| (mm) | | (mm) | | (mm) | | (mm) | | (mm) | | (mm) | |
| thickness | 1.49 | thick- | | triangle | 0.5 | thickness | | thick- | 0.448 | thick- | 0.618 |
| (mm) | | ness | | height | | | | ness | | ness | |
| | | (mm) | | (mm) | | | | (mm) | | (mm) | |
| | | | | thickness | 0.468 | | | | | | |
| | | | | (mm) | | | | | | | |
| Volume | 16.762 | | 2.646 | | 1.9305 | | 6.561 | | 2.016 | | 1.854 |
| (mm³) | | | | | | | | | | | |
| Surface | 5 | | 16.9440 | | 12.1356 | | | | 13.349 | | |
| Area | 45.544 | | 9622 | | 99 | | 26.748 | | 2 | | 10.326 |
| (mm²) | 1 | | 6.40366 | | 6.28629 | | 4.0768 | | 7536 | | 5.56957 |
| SA to V | 2559 | | 4484 | | 8367 | | 1 | | 6.6216 | | 9288 |
| ratio | 2,7170 | | | | | | 7558 | | 6 | | 0.0014 |
| Beginning | 2 | | 0.0037 | | 0.0015 | | | | 4366 | | 0.00075 |
| | 4644 | | 0.00339 | | 0.00077 | | 0.0034 | | | | 5124 |
| Mass (g) | | | 8337 | | 7001 | | 0.0005 | | 0.002 | | 10.0714 |
| Density | 0.0177 | | 7.78378 | | 11.2666 | | 1 | | 0.0009 | | |
| (g/mm*) | 0.0010 | | 3784 | | 6667 | | 8214 | | 9 | | 2857 |
| 5 min | 5 | | | | | | | | 2063 | | |
| swell | 5928 | | | | | | 9 | | broke | | |
| Ratio | 2.5480 | | | | | | | | before | | |
| | 2 | | | | | | | | 5 min | | |
| | 2599 | | | | | | | | | | |

Swelling data for the various formulations is graphed in FIG. 14A (swelling within 5 min) and FIG. 14B (swelling within 60 min).

As can be seen from the primary data, the quickest swelling gel was gel No. 23, which swelled 2000% in 5 min, which compares quite well to the 300% swelling rate for polyacrylamide gels. When allowed to swell for 60 min, gel No 19 swelled nearly 7000%, while gel No. 23 swelled 4000%.

As the ideal gel has rapid swelling and reaches its maximum swelling state quickly, gel No. 23 is the best gel based on swelling data alone. Gel No. 23 consists of 15% Acrylic acid and 0.05% poly(ethylene glycol) diacrylate. Gel No. 19 consists of 10% Acrylic acid and 0.05% poly(ethylene glycol) diacrylate.

### Example 2

### Assessment of Alternative Crosslinkers for Hydrogels

The principle behind the selected crosslinkers is that rather than having a short cross-linker with only two polymerizable groups, a polyvalent crosslinker (i.e., a long-chain hydrophilic polymer with multiple polymerizable groups) is being used. A much stronger hydrogel is obtained compared to short chain, divalent crosslinkers. While these gels are very firm, they possess very good swelling characteristics. Very strong gels do not normally swell very much.

Poly vinyl alcohol (PVA) was derivatized with allyl glycidyl ether under alkaline conditions. Gels were made by combing acrylic acid with the PVA-based crosslinker and then polymerizing the mixture by free radical polymerization using ammonium persulfate and TEMED as initiators.

In principle, the crosslinker can be made with a number of different starting materials: A range of PVAs as well as partially hydrolyzed poly vinyl acetates, 2-hydroxyethyl methacrylates (HEMA) or various other polymers with reactive side groups can be used as the basic polymeric backbone. In addition, a wide range of natural hydrocolloids such as dextran, cellulose, agarose, starch, galactomannans, pectins, hyaluronic acid etc. can be used. A range of reagents such as allyl glycidyl ether, allyl bromide, allyl chloride etc. can be used to incorporate the necessary double bonds into this backbone. Depending on the chemistry employed, a number of other reagents can be used to incorporate reactive double bonds.

Preparation of Polyvalent Crosslinker

Polyvinyl alcohol (PVA, 30-70 kDa) was derivatized with allyl glycidyl ether under alkaline conditions. 2 g PVA was dissolved in 190 mL water. Once fully dissolved, 10 mL 50% NaOH was added, followed by 1 mL allyl glycidyl ether and 0.2 g sodium borohydride. The reaction was allowed to proceed for 16 hours. Subsequently, the crosslinker was precipitated from the reaction mixture by addition of isopropanol. The precipitate was collected by filtration, washed with isopropanol, and re-dissolved in 50 mL of water. The crosslinker was utilized for gel formation, as described below without further purification or characterization.

Gel Formation and Characterization

Gels were formed by combining acrylic acid with the PVA-based crosslinker prepared above, and then polymerizing the mixture by free radical polymerization using ammonium persulfate and TEMED as initiators.

Three gels were prepared containing 15% acrylic acid in combination with various ratios/concentrations of the PVA-based crosslinker. The components listed in Table 3 (excluding initiators) were mixed and degassed by placing the tubes in a desiccator with a vacuum applied. After 10 minutes, the vacuum was turned off, and the tubes remained in the desiccator for a further 10 minutes under vacuum. The desiccator was opened, and the initiator was added. The contents of the tubes were then mixed thoroughly. The tubes were capped and left overnight to polymerize, forming hydrogels.

TABLE 3
Composition of gels 23a-c formed using
polyvalent PVA-based crosslinkers.

| | Gel | | |
|---|---|---|---|
| Components (mL) | 23a | 23b | 23c |
| acrylic acid | 1.5 | 1.5 | 1.5 |
| PVA cross-linker | 0.0526 | 0.526 | 5.26 |
| 50% NaOH | 1.251 | 2.15 | 2.35 |
| H2O | 7.122 | 5.779 | 0.795 |
| APS | 0.04 | 0.04 | 0.04 |
| TEMED | 0.05 | 0.05 | 0.05 |
| total | 10.02 | 10.05 | 10.00 |
| pH (pre-initiator addition) | 7.416 | 7.557 | 7.451 |

[Please Provide any Further Details You have Related to these Gels—C_{g} Values, T_{g} Values, etc.]

The gel had a faint pink color, and exhibited a pH of approximately 7.7 when gelled. An increase in opacity in the gels was observed, with gel 23a having the lowest opacity, and gel 23c having the highest opacity. The gels had gel strength that was significantly higher than the gels made with the poly(ethylene glycol) diacrylate as crosslinker. The gels had very good mechanical properties as well as very good swelling. The swelling rates for gels 23a-c were measured, and are shown in Table 4. Percent swelling was measured after 5 minutes and 60 minutes.

TABLE 4
Swelling behavior of gels 32a-c formed using
polyvalent PVA-based crosslinkers.

| Gel | 23a | 23b | 23c |
|---|---|---|---|
| 5 min swelling* | 1000-2000% | 250-1100% | 900-1000% |
| 60 min swelling* | 4000-6000% | 1100-2500% | 3600-4300% |

*3 repeats were made for each gel swelling experiment

The purpose of this work was to provide proof of concept for new gel types. These included gels with different type of cross-linkers that consist of long polymer chains with multiple reactive groups, gels anchored to a substrate, and sponges. The idea behind the new cross-linker types is that long-chain cross-linkers and cross-linkers with multiple double-bonds could result in more resilient gels with better long term durability than gels utilising smaller cross linkers with only two double-bonds. The project was conducted in two phases with an initial phase that showed that functional, long chain cross-linkers with multiple double-bonds could be made and that gels could also be made with these types of cross-linkers. The project then proceeded to the second phase, which aimed at getting more detailed information about the properties of gels made with these cross-linkers by making further variations of cross-linkers and gels. The properties of the gels with respect to swelling rates, swell force and durability were investigated.

Formulation and manufacture of the gels was performed by IPT Pty. Ltd., while testing of the gels was performed by Endoluminal Sciences Pty. Ltd.

### Cross-Linker Preparation

The use of various cross-linkers was explored, both in terms of cross-linker type and amount used in a formulation. The starting materials are listed below.

### Materials and Equipment

Polyvinyl alcohol (MW 30-70 000)—Sigma, PN P8136.

Polyvinyl alcohol (MW 89-98 000)—Sigma, PN 341584.

Polyvinyl alcohol (App. MW 100 000)—Sigma, PN P1763.

Carboxymethylcellulose—Aqualon, 7MF.

Allylglycidyl ether—Sachem, ZBU-182680.

19M NaOH for pH adjustment.

Bromine water—Labtek, PN AG00050500.

Isopropyl alcohol for rinsing.

Magnetic stirrer and stirrer bars.

Balance.

Fume hood.

Pipettes—100 μL.

Beakers and measuring cylinders.

Method—

Preparation of PVA for Gel Formulation

The polyvinyl alcohol (PVA) materials were activated with allylglycidyl ether (AGE) at various levels and reaction times. This was also done for the carboxymethyl cellulose (CMC). The required amount of PVA (6 g) was weighed into a beaker and deionized (DI) water was added to make the mixture up to just under 60 mL. The CMC experiments required 300 mL for 6 g. The appropriate amount of AGE was then added while the mixture was being stirred with a magnetic stirrer bar. NaOH was then added to a concentration of 0.2M. The final volume of the mixture was then adjusted to 60 mL. The mixture was allowed to react before being neutralised, rinsed with isopropyl alcohol and dried under vacuum. A table of all PVA and CMC materials is provided in the results section.

Method—Measurement of AGE Incorporation

Solutions of the modified PVA/CMC materials were prepared in deionized (DI) water and heated until fully dissolved. A sample of the prepared solution (either 1 or 2 mL) was titrated against the bromine water in order to determine the number of double bonds present and thus the level of incorporation. A table of results is provided in the results section.

### Gel Manufacture

The cross-linkers prepared above were used with acrylic acid to prepare various gels. In addition to these, gels were prepared with both poly(ethylene glycol) diacrylate (PEG) or (BIS) for comparison with known gel formulations.

### Materials and Equipment

Acrylic acid—Sigma, PN 147230.
Poly(ethylene glycol) diacrylate—Sigma, PN 455008.
N,N′-methylene-bisacrylamide (BIS)—Sigma, PN 146072.
Cross-linkers described above in section 2.
Ammonium persulfate(APS)—Sigma, PN A9164.
N,N,N′,N′-tetramethylethylenediammine(TEMED)—Sigma, PN T9281.
19M NaOH for pH adjustment.

### Sigmacote—Sigma, PN SL2.

Glass plates—15×15 cm.
Plastic spacers—20×1×0.5 cm.
“Bulldog” clips.
Magnetic stirrer and stirrer bars.
Desiccator with vacuum attachment.
High vacuum pump.
pH meter (calibrated before use).

### Balance.

### Pipettes—1000 to 20 uL.

Beakers and measuring cylinders.
Vacuum dryer.
Fabric support (provided by Endoluminal Sciences)

Method—Preparation of Glass Plates

The glass plates are first prepared by washing them in hot soapy water and rinsing thoroughly with tap and then DI water. The glass plates are then wiped with IPA to remove any dirt or grease not removed by the previous rinsing. A few millilitres of Sigmacote is added to the surface of each glass plate and then wiped over the entire surface with a piece of paper towel, ensuring all areas of the plate are covered. The plates are left to completely dry overnight before use.

Method—Gel Casting

The required amounts of acrylic acid and cross-linker are added to an 80 mL beaker. DI water is added to make the volume approximately 75% of the final volume. The pH of the solution is then measured and recorded. The solution is then adjusted to a pH value close to 7.4 with 19M NaOH (typically 8 mL is required for a 40 mL solution). The solution volume is then adjusted with DI water to the final required volume and the pH measured again and recorded (measuring cylinder). Minor adjustments to the pH can then be made with either NaOH or HCl if required. The solution is then transferred to the beaker with a stirring bar. The beaker is then placed in the desiccators, stirrer started and the vacuum applied to the system in order to remove as much dissolved oxygen as possible. The vacuum is applied for 30 minutes. The glass plates are assembled so that the treated surface is facing up and then two spacers are placed on the surface. The initiator solutions are prepared by making 20% solutions of APS and TEMED in DI water. Once the 30 minutes vacuum has been completed, the stirrer is turned off and the vacuum released. The stirrer is then turned on as a low speed and as quickly as possible the TEMED solution is added followed by the APS solution. Still working as quickly as possible, approximately 25 mL of solution is poured onto the centre of the prepared glass plate. Another glass plate is carefully placed on top of the solution (treated side facing the solution) taking care not to include any air bubbles. The assembly is them held in place by clamping the edges with “bulldog” clips and left overnight to cure. The remaining solution is then placed in a labelled tube to confirm gelling.

Method—Gel Drying

The following day the top glass plate is removed and an appropriate piece of fabric support is placed onto the gel surface. The gel is removed from the other glass plate by gentle peeling the gel off the glass, leaving the gel on the fabric. The fabric is then placed on a vacuum dryer and dried for 95 minutes, 60 minutes of which include heating to 40° C. The dried gel is then handed over to Endoluminal Sciences for testing.

### Gel Testing

Swell Rate—Materials, Method and Apparatus

Hydrogel sample.
Metal containers and sieves.
Water bath set to 37° C.

### 1 L of PBS at 37° C.

### Balance.

### Timer.

Set water bath to 37° C. and prepare PBS at 37° C. Prepare three 1 cm×1 cm samples of hydrogel and weigh (initial weight). Place each sample into a metal container and place the container in the water bath. Half fill the containers with PBS at 37° C. and leave for 1 hr. Separate the hydrogel from the PBS with the sieve and blot dry with paper towel. Weigh each sample (final weight).

Stress and Strain—Materials, Method and Apparatus

Hydrogel sample.
Cylinder and piston jig.

### PBS at 37° C.

### ELS Tensile Machine.

50N force gauge.

Take swollen hydrogel sample and stamp out three 10 mm diameter discs. Insert the three discs into the cylinder, ensuring that they are flat against the bottom of the supporting mesh. Attach the piston to the force gauge and then place the cylinder under the piston. Lower the piston down into the cylinder until the hydrogel and piston faces are 5 mm apart. Perform the compression test and record data for the stress-strain curve.

Maximum Swell—Materials, Method and Apparatus

Hydrogel sample.

### PBS at 37° C.

### ELS Tensile Machine.

50N force gauge.

A 10 mm diameter piece of dry hydrogel is stamped out and the thickness and weight measured. Set water bath to 37° C. and prepare PBS at 37° C. Dry the piston and cylinder thoroughly with hot air. Attach the piston to the force gauge and place the hydrogel into the cylinder. Place the cylinder on the piston and the metal container under the cylinder. Decrease the distance of the tensile machine block so that the force acting on the hydrogel is 0.2N. Place the tensile machine assembly in the water bath and ensure that the water level is below the lip of the metal container. Adjust the force gauge sampling rate to “fast” so that it takes a reading every 0.152 s. Activate force recording on the PC. Add the warm PBS into the metal container and start recording data. Wait for 1 hour, ensuring that data is being recorded. Stop the experiment and retrieve the swollen hydrogel. Remove excess PBS with paper towel and record the final weight of the sample.

Durability—Materials, Method and Apparatus

Hydrogel sample.
Durability test jig.
Water bath set at 37° C.

### 1 L of PBS at 37° C.

Prepare three swollen hydrogel samples by stamping out a 10 mm diameter disc. Layer the three samples into the graft pocket. Place sample into the durability jig, gluing the graft pocket to the underside. Place jig in hot water bath, ensuring that the lubricating pump nozzle is placed over the cam. Turn durability test jig on for 24 hrs. After this time, remove the gels from the pocket and visually check for cracks, tears and deformation of the sample. Carry out stress/strain test.

| | | | AGE level | |
|---|---|---|---|---|
| | | | (μL/g | Incorporation |
| Batch | | Reaction | cross- | (mol/mol |
| # | Cross-linker | time (hr) | linker) | sample) |
| ELS020 | PVA (MW 100 000) | 16 | 100 | — |
| ELS021 | PVA (MW 30-70 000) | 16 | 100 | — |
| ELS025 | PVA (MW 100 000) | 1.5 | 100 | 52 |
| ELS026 | PVA (MW 30-70 000) | 1.5 | 100 | 11 |
| ELS029 | PVA (MW 100 000) | 1.5 | 300 | 31 |
| ELS030 | PVA (MW 30-70 000) | 1.5 | 300 | 36 |
| ELS035 | CMC (MW 250 000) | 1.5 | 300 | 52 |
| ELS045 | PVA (MW 100 000) | 1.5 | 600 | 21 |
| ELS046 | PVA (MW 89-98 000) | 1.5 | 600 | 21 |

### Results and Observations

### Cross-Linkers

Batches ELS020 and ELS021 gradually changed to a dark brown colour over the 16 hours. It was decided to stop the subsequent reactions at 1.5 hours when the first signs of colour change occur.

Gels—PEG as Cross-Linker

This is the standard formulation in which it is used at a level of 1.5% w/w acrylic acid. It equates to a % T and % C of 15.2 and 1.4 respectively. The term % T in this case refers to the total amount of acrylic and cross-linker over the solution volume. The tem % C refers to the amount of cross-linker over the total amount of cross-linker and acrylic acid. Some gels were also made with half the level of initiators. This was done in an effort to produce a “softer” gel by having fewer new polymer chains cross-linking and thus, longer chains.

A summary of the results is shown in the table below.

| | % Swell in | Max. Swell | Stress/Strain | Initiator |
|---|---|---|---|---|
| Batch # | 1 hr | Force (N) | comments | Levels (mM) |
| ELS019 | 4997 | 9.9 | Initial fail at | 3.5 and 6.7 |
| | | | 20N | (usual) |
| ELS044 | 3013 | 6.3 | Before and | 3.5 and 6.7 |
| | | | after plots fail | (usual) |
| | | | at 50N | |
| ELS054 | 3119 | 7.5 | Before and | 1.8 and 3.3 |
| | | | after plots fail | (half) |
| | | | at 50N | |
| ELS056 | 2311 | 2.4 | Before plots | 1.8 and 3.3 |
| | | | fail at 15N, | (half) |
| | | | after plots fail | |
| | | | at 15-20N | |

ELS019 - initial testing indicate that the gel is quite brittle, initially yielding between 10 and 15N.
ELS044 - The stress/strain plots after the compression test are very similar to the before test results, indicating a very durable gel.
ELS054 - Before compression test.
The stress/strain plot for ELS054 (half initiators) is very similar to that of ELS044. After compression test the “after” plot is almost identical to the “before” plot, indicating that a reduction in initiators did not have a detrimental effect on the durability of the gel.
ELS056 - Before compression test.

The “before” plot is significantly different to that of ELS054, yielding at a much lower force. After compression test. The “after” plots are similar to the “before” plots indicating that the gels are still durable, albeit at a lower yield force. An observation made regarding the half initiators was that the colour of the residual gel for these gels was a beige-brown colour as opposed to the pink colour of the gels made with the standard amount of initiators.

Gels—BIS as Cross-Linker

This is a cross-linker that is widely used in polyacrylamide gel manufacture. It was used here as a comparison to the other cross-linkers at a level of 0.33% w/w acrylic acid which is the equivalent molar amount when compared with PEG. It equates to a % T and % C of 15.1 and 0.3 respectively. A summary of the results is shown in the table below.

| | % Swell in | Max. Swell | Stress/Strain | Initiator |
|---|---|---|---|---|
| Batch # | 1 hr | Force (N) | comments | Levels (mM) |
| ELS022 | 3149 | 9.8 | Before and | 3.5 and 6.7 |
| | | | after plots fail | |
| | | | at 10N | |

ELS022 - Before compression test the plots for ELS022 indicate a very brittle gel, yielding just before 10N.
ELS022 - After compression test the “after” plots are similar to the “before” plots, yielding just before 10N.

Gels—PVA as Cross-Linker

The various modified PVA materials were assigned codes for ease. These are in the table below.

| PVA | | Modified PVA | |
|---|---|---|---|
| code | Description | Batch # | Description |
| PVA1 | 5% solution of | ELS020 | PVA (MW 100 000), |
| | ELS020. | | reacted for 16 hrs with |
| | | | 100 μL/g of AGE. |
| PVA2 | 10% solution of | ELS021 | PVA (MW 30-70 000), |
| | ELS021. | | reacted for 16 hrs with |
| | | | 100 μL/g of AGE. |
| PVA3 | 2% solution of | ELS025 | PVA (MW 100 000), |
| | ELS025. | | reacted for 1.5 hrs with |
| | | | 100 μL/g of AGE. |
| PVA4 | 20% solution of | ELS026 | PVA (MW 30-70 000), |
| | ELS026. | | reacted for 1.5 hrs with |
| | | | 100 uL/g of AGE. |
| PVA5 | 5% solution of | ELS025 | PVA (MW 100 000), |
| | ELS025. | | reacted for 1.5 hrs with |
| | | | 100 μL/g of AGE. |
| PVA6 | 10.5% solution of | ELS026 | PVA (MW 30-70 000), |
| | (dried) ELS026. | | reacted for 1.5 hrs with |
| | | | 100 μL/g of AGE. |
| PVA7 | 5% solution of | ELS029 | PVA (MW 100 000), |
| | ELS029. | | reacted for 1.5 hrs with |
| | | | 300 μL/g of AGE. |
| PVA8 | 10% solution of | ELS030 | PVA (MW 30-70 000), |
| | ELS030. | | reacted for 1.5 hrs with |
| | | | 300 μL/g of AGE. |
| PVA9 | 5% solution of | ELS045 | PVA (MW 100 000), |
| | ELS045. | | reacted for 1.5 hrs with |
| | | | 600 uL/g of AGE. |
| PVA10 | 5% solution of | ELS046 | PVA (MW 89-98 000), |
| | ELS046. | | reacted for 1.5 hrs with 600 |
| | | | μL/g of AGE. |

Gels were made with the following PVA materials.

| | | Amount used | |
|---|---|---|---|
| Batch | PVA | (% w/w acrylic | |
| # | code | acid) | Comments |
| ELS023 | PVA1 | 1.3 | Dissolved in PBS after 1 hr. |
| ELS024 | PVA2 | 1.3 | Gel was very sticky and could |
| | | | not be transferred for drying |
| | | | and testing. |
| ELS027 | PVA3 | 4.1 | Dissolved in PBS after 1 hr. |
| ELS028 | PVA4 | 4.3 | Gel was very sticky and could |
| | | | not be transferred for drying |
| | | | and testing. |
| ELS031 | PVA5 | 8.3 | Dissolved in PBS after 1 hr. |
| ELS032 | PVA5 | 20.8 | Dissolved in PBS after 1 hr. |
| ELS033 | PVA6 | 36.2 | Gel was very sticky and could |
| | | | not be transferred for drying |
| | | | and testing. |
| ELS037 | PVA7 | 16.5 | Dissolved in PBS after 1 hr. |
| ELS038 | PVA8 | 16.5 | Gel was very sticky and could |
| | | | not be transferred for drying |
| | | | and testing. |
| ELS057 | PVA7 | 16.5 | Gel formulation was done at a |
| | | | pH of 5 rather than 7.4. A firm |
| | | | gel was produced. |

All of the gels made with just acrylic acid and any of the PVA materials were not able to be tested. Of the gels made, those that used the higher molecular weight PVA (100 000) were “firmer” than the lower molecular weight. From these results it was decided that a combination of PEG and PVA would be worth investigating.

For all the PVA formulations, the solution became more and more turbid as the pH of the gel solution was increased from 2 to 7.4. The gel made at a pH of 5 rather than 7.4 when the solution was just becoming turbid. This formulation was the exception and resulted in a firm gel.

Gels—PVA and PEG as Cross-Linkers

The formulations for the combined cross-linkers are given in the table below.

| | | Amount used | | |
|---|---|---|---|---|
| Batch | PVA | (% w/w acrylic | PEG (% w/w | |
| # | code | acid) | acrylic acid) | Comments |
| ELS036 | PVA5 | 8.3 | 0.75 | Firm, flexible gel. |
| ELS040 | PVA7 | 5.5 | 0.75 | Firm gel. |
| ELS042 | PVA7 | 8.3 | 0.9 | Firm gel. |
| ELS048 | PVA9 | 8.3 | 0.75 | Firm gel. |
| ELS049 | PVA10 | 8.3 | 0.75 | Firm gel. |

| | % Swell in | Max. Swell | |
|---|---|---|---|
| Batch # | 1 hr | Force (N) | Stress/Strain comments |
| ELS036 | 3149 | 9.8 | Before and after plots fail |
| | | | before 10N. |
| ELS040 | 4074 | 7.5 | Before plot failed at 20- |
| | | | 25N, after plot failed at |
| | | | 30N. |
| ELS042 | 2684 | 16.3 | Before plot failed at 35- |
| | | | 50N, after plot failed at |
| | | | 10-25N. |
| ELS048 | 2672 | 11.6 | Before plot failed at 30N, |
| | | | after plot failed at 35. |
| ELS049 | 2959 | 2.6 | Before plot failed at 20N, |
| | | | after plot failed at 25-30. |

ELS036 - Before compression test was 8.3% PVA5 and 0.75% PEG (of acrylic acid).The “before” plot failed before 10N indicating that it was a brittle gel. ELS036 - After compression test the “after” plot was slightly better than the “before”.
ELS040 - Before compression test ELS040 was 5.5% PVA7 and 0.75% PEG (of acrylic acid). The “before” plot failed before 10N indicating that it was a brittle gel. ELS040 - After compression test the “after” plot was slightly better than the “before”.
ELS042 - Before compression test was 8.3% PVA7 and 0.9% PEG (of acrylic acid). The “before” plot failed between 35 and 50N which is an improvement over ELS040. ELS042 - After compression test the “after” plot did not perform as well as the “before”, failing at approximately 20N. Despite the improved durability of the initial testing, the increase in both PVA7 and PEG did not result in a more durable gel.
ELS048 - Before compression test was 8.3% PVA9 and 0.75% PEG (of acrylic acid). The “before” plot failed at App. 30N which indicates that it is less brittle than ELS036 which used PVA5 at the same level. ELS048 - After compression test the “after” plot performed slightly better than the “before”, failing at approximately 35N. The use of PVA9 instead of PVA5 resulted in a more durable gel. Both of the modified PVA's used the 100 000MW as the starting material but PVA9 was activated with AGE at a higher level, 600 uL/g compared with 100 uL/g for PVA5.
ELS049 - Before compression test was 8.3% PVA10 and 0.75% PEG (of acrylic acid). The “before” plot failed at App. 20N which indicates that it is slightly more brittle than ELS048. After compression test the “after” plot performed slightly better than the “before”, failing at 25-30N. The PVA used in PVA9 (ELS048) was the high molecular weight (App. 100 000). The PVA used in PVA10 (ELS049) has a narrower molecular weight range (89-98 000). Both were activated with 600 uL/g AGE. The slightly lower results for ELS049 compared with ELS048 indicates that the higher molecular weight PVA is preferable in terms of gel durability.

Gels—CMC and PEG as Cross-Linkers

The formulations for the combined cross-linkers are given in the table below.

| | | Amount used | | |
|---|---|---|---|---|
| Batch | CMC | (% w/w acrylic | PEG (% w/w | |
| # | code | acid) | acrylic acid) | Comments |
| ELS039 | CMC1 | 10.3 | 0 | Not testable but |
| | | | | did not dissolve. |
| ELS041 | CMC1 | 5.2 | 0.75 | Firm gel. |
| ELS047 | CMC1 | 3.9 | 0.75 | Firm gel. |

| | % Swell in | Max. Swell | |
|---|---|---|---|
| Batch # | 1 hr | Force (N) | Stress/Strain comments |
| ELS039 | — | — | — |
| ELS041 | 3366 | 10.9 | Before plot failed at 20N, |
| | | | after plot failed at 10N. |
| ELS047 | 3393 | 10.7 | Before plot failed at 20N, |
| | | | after plot failed at 30N. |

ELS041 - Before compression test was 5.2% CMC1 and 0.75% PEG (of acrylic acid). The “before” plot failed at App. 20N. After compression test the “after” plot performed worse than the “before” failing at 10N, indicating that the CMC gel was less durable than the PVA based gels at that level of CMC.
ELS047 - Before compression test was 3.9% CMC1 and 0.75% PEG (of acrylic acid). The “before” plot failed at App. 20N. After compression test the “after” plot performed better than the “before” failing at 30N, indicating that by reducing the CMC level a more durable gels could be formed.

Gels—Substrates

The standard formulation was cast onto a piece of Gel-Fix for PAG (Serva, PN 42980). The first attempt failed to adhere to the substrate. The second attempt was more successful and the gel did adhere to the substrate after casting but started to come off when vacuum dried. These formulations were not tested (ELS043 and ELS052). Similarly, the standard formulation was cast directly onto the fabric used to support the gels during vacuum drying (ELS053 and ELS055). The gel became “crinkled” after swelling in PBS for 1 hour and fell off the support when moved. The gel was not tested further.

Discussion

A table containing a data and comment summary of all the gels made is provided below. The work showed that making gels with the new types of cross-linkers is not only possible, but that there may also be scope for obtaining gels with better properties than the current gels, especially with respect to durability. More variation than expected was seen in the testing of repeated gel formulations, and a number of contributing factors are believed to play a role, including:

Oxygen concentration and pH in the gel preparations before casting

storage of dried gels before testing

testing of gels with variable moisture content

Specific points to the individual gel types are:

PEG as Cross-Linker

There was some variation in all parameters for this standard formulation. Even given the level of variation, the swell force results were too high. The work with alternative cross-linkers addressed this.

Formulations with half the level of initiators had a different colour and a slightly softer gel. This suggests that at the usual level of initiators, the initiators are “mopping up” excess oxygen rather than taking part in polymerisation. The two points above suggest that the effect of oxygen in the solution on the subsequent gel is substantial.

PVA as Cross-Linker

The gels made using the different types of PVA (MW of 30-70 000, App. 100,000 and 89-98,000 at various levels of AGE activation) did not make “testable” gels, being either too sticky and hard to handle or “dissolving” in PBS after 1 hr. The increasing levels of AGE activation were tried in order to improve the durability of the gels by providing additional sites for cross-linking This could not be tested as the resultant gels still “dissolved” in PBS or were too sticky.

When increasing the pH from a starting point of 2 to 7.4, all of the PVA solutions became turbid as the PVA came out of solution. An attempt to prevent this was made by adjusting the pH to 5. This provided a firm gel with good swell characteristics using PVA with a molecular weight of App. 100 000 and activated using 300 uL/g of AGE. It is possible that some of the other PVA modifications would also provide good gels if cast at the lower pH.

PVA and PEG as Cross-Linkers

The combination of PVA and PEG as cross-linkers provided firm gels with good properties when tested. As stated above, the increasing levels of AGE activation were tried in order to improve the durability of the gels by providing additional sites for cross-linking. This proved to be the case when using the App. 100 000 MW PVA with 100, 300 and 600 uL/g AGE. There was an increase in the stress/strain failure force after the durability test (ELS036 versus ELS042 versus ELS048).

CMC and PEG as Cross-Linkers

The use of CMC was inspired by the prospect of having even more “active sites” for cross-linking than PVA. The results were similar to those using PVA/PEG.

Substrates

Poor results were achieved when casting gels directly onto the fabric. The activated substrate gave poor results as well. Better results may be achieved by using a different brand/type of activated substrate.

| | | | | max. | | |
|---|---|---|---|---|---|---|
| | | | | swell | | |
| | | | % | force | | |
| Gel | Cross- | ELS general | swell | 1 hr | | |
| code | linker | comments | (1 hr) | (N) | Stress/Strain | comments |
| ELS019 | PEG | Brittle. | 4997 | 9.9 | Initial fail at | Firm gel. Vacuum dried. |
| | | | | | 20N. No | |
| | | | | | “after”plot. | |
| ELS022 | BIS | Can be stretched, not | 3149 | 9.8 | Before and | Firm gel. Vacuum dried (1.5 hrs |
| | | brittle. | | | after fail at | longer than usual). |
| | | | | | 10N. | |
| ELS023 | PVA1 | Dissolved in PBS after | — | — | — | Gel was sticky. Vacuum dried as |
| | | 1 hr. | | | | for ELS022 with gel still on the |
| | | | | | | glass plate. Equivalent # moles as |
| | | | | | | used for PEG. |
| ELS024 | PVA2 | — | — | — | — | Gel was very sticky and not able to |
| | | | | | | be tested (not vacuum dried). |
| | | | | | | Equivalent # moles as used for |
| | | | | | | PEG. |
| ELS027 | PVA3 | Dissolved in PBS after | — | — | — | Gel was sticky (similar to ELS023). |
| | | 1 hr. | | | | Vacuum dried as for ELS022. |
| | | | | | | Equivalent # moles as used for |
| | | | | | | PEG. |
| ELS028 | PVA4 | — | — | — | — | Gel was very sticky and not able to |
| | | | | | | be tested (not vacuum dried). |
| | | | | | | Equivalent # moles as used for |
| | | | | | | PEG. |
| ELS031 | PVA5 | Dissolved in PBS after | — | — | — | Gel was sticky but some was |
| | | 1 hr. | | | | transferred to a sheet for testing. |
| | | | | | | Vacuum dried for 2 hrs (1 hr @ |
| | | | | | | 40° C.). Twice the equivalent # |
| | | | | | | moles as used for PEG. |
| ELS032 | PVA5 | Dissolved in PBS after | — | — | — | Gel was slightly sticky but some |
| | | 1 hr. | | | | was transferred to a sheet for |
| | | | | | | testing. Vacuum dried for 2 hrs (1 hr |
| | | | | | | @ 40° C.). 4x the equivalent # moles |
| | | | | | | as used for PEG. |
| ELS033 | PVA6 | — | — | — | — | Gel was quite sticky and not easily |
| | | | | | | transferred to testing. 5x the |
| | | | | | | equivalent # moles as used for |
| | | | | | | PEG. |
| ELS036 | PEG/PVA5 | Flexible, residue left on | 2517 | 7.3 | Before plots | Gel was firm and easily removed |
| | | finger when touched. | | | fail 5-10 N, | from the plate. The gel was not |
| | | After vacuum drying, | | | “after” | vacuum dried but air dried for 3 hrs. |
| | | consistent swell | | | slightly | |
| | | achieved. | | | better at 10 N. | |
| ELS037 | PVA7 | Dissolved in PBS after | — | — | — | Gel was firmest of all PVA gels so |
| | | 1 hr. | | | | far. The gel was not vacuum dried |
| | | | | | | but air dried for 3 hrs. 4x the |
| | | | | | | equivalent # moles as used for |
| | | | | | | PEG. |
| ELS038 | PVA8 | — | — | — | — | Gel was very sticky and could not |
| | | | | | | be separated in one piece from the |
| | | | | | | plates. 4x the equivalent # moles as |
| | | | | | | used for PEG. |
| ELS039 | CMC1 | Could not form a | — | — | — | Gel was quite firm and easily |
| | | testable sheet but did | | | | removed from the glass. Equivalent |
| | | not dissolve. | | | | # moles as used for PEG. |
| ELS040 | PEG/PVA7 | No slippery feeling, | 4074 | 7.5 | Before plot | Gel was firm and easily removed |
| | | quite firm. After further | | | failed at 20-25, | from the plate. The gel was vacuum |
| | | drying, increase in swell | | | after | dried (1 hr @ 40° C., 1.5 hrs |
| | | rate. | | | plots at 30. | vacuum). Half the usual amount of |
| | | | | | | PEG was used. |
| ELS041 | PEG/CMC1 | No slippery feeling, | 3366 | 10.9 | Before plot | Gel was firm and easily removed |
| | | quite firm. | | | failed at 20, | from the plate. The gel was vacuum |
| | | | | | after plots at | dried (1 hr @ 40° C., 1.5 hrs |
| | | | | | 10. | vacuum). Half the usual amount of |
| | | | | | | PEG was used. |
| ELS042 | PEG/PVA7 | No slippery feeling, | 2684 | 16.3 | Before plot | Gel was firm and easily removed |
| | | very firm and tough. | | | failed at 35-50, | from the plate. The gel was vacuum |
| | | | | | after | dried (1 hr @ 40° C., 1.5 hrs |
| | | | | | plots at 10-25. | vacuum). 75% the usual amount of |
| | | | | | | PEG and 50% more PVA7 than |
| | | | | | | ELS040 was used. |
| ELS043 | PEG | Dissolved in PBS after | — | — | — | Gel was firm. It did not stick to the |
| | | 1 hr. | | | | gel bond as expected after vacuum |
| | | | | | | drying. |
| ELS044 | PEG | Swell force only reach 6 N | 3013 | 6.3 | Before and | Gel was firm and came off the glass |
| | | in one hour, reach | | | after plots | plate easily. The gel was vacuum |
| | | 11 N in 17 hours. | | | failed at 50. | dried (1 hr @ 40° C., 1.5 hrs |
| | | ELS019 reached 10 N in | | | | vacuum). |
| | | one hour | | | | |
| ELS047 | PEG/CMC1 | Ordinary gel | 3393 | 10.7 | Before plot | Firm gel. |
| | | | | | failed at 20, | |
| | | | | | after plots at | |
| | | | | | 30. | |
| ELS048 | PEG/PVA9 | Ordinary gel | 2672 | 11.6 | Before plot | Firm gel (slightly opaque). |
| | | | | | failed at 30, | |
| | | | | | after plots at | |
| | | | | | 35 (variable). | |
| ELS049 | PEG/PVA10 | Ordinary gel | 2959 | 2.6 | Before plot | Firm gel (slightly opaque). |
| | | | | | failed at 20, | |
| | | | | | after plots at | |
| | | | | | 25-30. | |
| ELS050 | PEG | — | — | — | — | Firm gel (problems with vacuum |
| | | | | | | pump when drying). |
| ELS051 | PEG | — | — | — | — | Half the initiators used. Firm gel |
| | | | | | | (problems with vacuum pump when |
| | | | | | | drying). |
| ELS052 | PEG | — | — | — | — | Gel-Fix used as substrate/support. |
| | | | | | | Gel was firm and adhered but came |
| | | | | | | off when vacuum dried. |
| ELS053 | PEG | After 1 hr swell, gel is | — | — | — | Gel cast with fabric support in |
| | | swollen, and became | | | | place. |
| | | crinkle Swollen | | | | |
| | | crinkled gel weakly | | | | |
| | | stuck on the mesh. | | | | |
| | | When the mesh is lifted | | | | |
| | | up, HG fell down. | | | | |
| ELS054 | PEG | Lots of cracks. | 3119 | 7.5 | Before and | Half the initiators used. |
| | | | | | after plot | |
| | | | | | failed at 50. | |
| ELS055 | PEG | | — | — | — | Gel cast with fabric support in |
| | | | | | | place. |
| ELS056 | PEG | Sticky feeling on | 2311 | 2.4 | Before plot | Half the initiators used. |
| | | surface after swollen. | | | failed at 15, | |
| | | | | | after plots at | |
| | | | | | 15-20. | |
| ELS057 | PVA7 | | 2556 | 3.3 | Before plot | Solution pH was 5 rather than 7.4. |
| | | | | | failed at 45-50, | |
| | | | | | after | |
| | | | | | plots at 50. | |

From the foregoing, it will be appreciated that specific embodiments of the disclosure have been described herein for purposes of illustration, but that various modifications may be made from these embodiments. Certain aspects of the disclosure described in the context of particular embodiments may be combined or eliminated in other embodiments. For example, a sealing device in accordance with particular embodiments may include only some of the foregoing components and features, and other devices may include other components and features in addition to those disclosed above. Further, while advantages associated with certain embodiments have been described in the context of those embodiments, other embodiments may also exhibit such advantages, and not all embodiments need necessarily exhibit such advantages. Accordingly, the disclosure can include other embodiments not shown or described above.

We claim:

1. An endoluminal seal for sealing an endoluminal implant or prosthesis to a wall of a lumen of a subject, the endoluminal seal comprising:
An expandable material selected from the group consisting of hydrogels, sponges and foams optionally spray dried or chemically coupled to the interior of the endoluminal seal,
A first membrane adjacent to and containing the expandable material;
Wherein the expandable material is activated by exposure to a fluid or a foaming agent.

2. The endoluminal seal of claim 1 wherein
the expandable material is a hydrogel which expands two to one hundred fold, preferably 50 to 90 fold, upon contact with a fluid, and
the first membrane is permeable to fluid.

3. The endoluminal seal of claim 2 comprising a swellable hydrogel material selected from the group consisting of polyacrylic acids and polyalkylene oxides.

4. A hydrogel for use in an endoluminal seal, wherein the hydrogel is able to expand rapidly upon hydration to at least ten times the volume of the dry state, more preferably up to 50× the volume of the dry state.

5. The hydrogel of claim 4 wherein the swelling force exerts a radial force between 0.001N/mm² and 0.025N/mm²′, more preferably between 0.008N/mm² and 0.012N/mm².

6. The hydrogel of claim 4 wherein the hydrogel expands from 2-100 times, preferably 50-90 times, most preferably about 60 times the volume of the dry state within 10 minutes, preferably less than 3 minutes, following contact with aqueous fluid.

7. The hydrogel of claim 4, wherein the hydrogel does not change volume at both room temperature and 37-40° C.

8. The hydrogel of claim 4, wherein the hydrogel comprises a long chain cross-linker having more than 20 carbons and/or a molecular weight greater than 400 Da, more preferably more than 40 carbon atoms and/or a molecular weight greater than 800 Da

9. The hydrogel of claim 8, wherein the long chain crosslinker is selected from the group consisting of polyvinyl alcohol, polyethylene glycol, polyvinyl acetate, dextrans, hyaluronic acids, agaroses, collagen, and starch.

10. The hydrogel of claim 8, wherein the crosslinker has multiple polymerizable groups.

11. The hydrogel of claim 10, wherein the multiple polymerizable groups are vinyl groups.

12. The hydrogel of claim 4, wherein the polymer is selected from the group consisting of acrylic acid, acrylamide or other polymerizable monomers; cross-linkers such as polyvinyl alcohols and partially hydrolyzed poly vinyl acetates, 2-hydroxyethyl methacrylates or other polymers with reactive side groups such as acrylic, allylic, and vinyl groups.

13. A fluid isolatable expandable seal for a vascular device comprising
A hydrogel strip,
A polymeric film encapsulating the hydrogel strip,
The encapsulated strip being positioned on the exterior circumference of the vascular device, wherein the exterior of the encapsulated strip expands upon contact with a fluid, and
The film has a slit that opens to allow fluid to hydrate the hydrogel strip.

14. The seal of claim 13 comprising a porous mesh between the hydrogel strip and the film.

15. The seal of claim 13 made by blow molding.

16. A fluid isolatable expandable seal for a vascular device comprising
A polymeric film expanding to form a seal which is positioned on the exterior circumference of the vascular device, wherein the exterior of the encapsulated strip expands upon contact with a fluid, and
The film has an opening that is open to allow fluid to fill the expandable film, which self-seals by positive displacement when the expandable film is fully hydrated.

17. The seal of claim 16 further comprising a hydrogel strip within the expandable film, which hydrates and expands when the film fills with fluid.

18. The seal of claim 16 further comprising a one-way valve which closes the expandable film when fully expanded.

19. A fluid isolatable expandable seal for a vascular device comprising
A polymeric film blow molded to form a “D” balloon over a porous mesh, which is heat or laser welded to seal a hydrogel strip between the film and the mesh.

20. The seal of claim 19 attached to the exterior circumference of a vascular device.

21. The seal of claim 19 having a cross sectional profile in the shape of the letter “D”, with the flat portion lying in abutment to the vascular device. prosthesis while the curved portion of the D profile faces outward.

22. The seal of claim 19 further comprising one or more engagement members.

23. A stent-balloon-vascular device with seal comprising
A stent containing a vascular device and balloon for centering of the device as it is positioned,
a seal on the inside of the stent containing the vascular device,
wherein the seal is positioned in abutment with the device, so that the seal is flipped out and over the end edge of the vascular device as the device is expanded and immediately prior to positioning, wherein the device is centered by the balloon.

24. The device of claim 23 comprising straps to flip the seal out and over the end edge of the device.

25. A removable casing formed of a metal or polymer for fluidic isolation of an expandable seal on the exterior of a vascular device,
the casing having as in a “U” shape that allows for complete insertion of the seal within the “U” cavity when attached to the exterior circumference of the vascular device,
wherein the open end of the “U” cavity has O-rings and a locking mechanism that fit together to compress the O-rings to bring them under pressure, thereby allowing the formation of a fluid-tight seal.

26. An endoluminal seal for sealing an expandable endoluminal implant or prosthesis to a wall of a lumen of a subject, the endoluminal seal comprising:
an expandable film containing a foaming material activatable by exposure to a fluid or a foaming agent, secured to the exterior circumference of a vascular device, the vascular device having on the interior circumference spring struts which push through the vascular implant or prosthesis to force the foam within the expandable material outward from the vascular device.

27. An endoluminal seal for sealing an expandable vascular device, the endoluminal seal comprising:
a polymeric fluid impermeable membrane removable casing formed of a metal or polymer for fluidic isolation of an expandable seal on the exterior of a vascular device,
an expandable material selected from the group consisting of hydrogels, sponges and foams optionally spray dried or chemically coupled to the interior of the endoluminal seal,
a first membrane adjacent to and containing the expandable material;
wherein the expandable material is activated by exposure to a fluid or a foaming agent, and
a fluid impermeable membrane fluidically isolating the expandable material until exposed to an aqueous solution under physiological conditions.

28. The seal of claim 27 wherein the fluid impermeable membrane remains intact in glutaraldehyde.

29. The seal of claim 27 wherein the membrane is made of polyvinyl alcohol or polyacrylamide which dissolves at physiological pH, in isotonic fluid, or in a specific liquid.

30. A plug for preventing exposure of an endoluminal seal for sealing an expandable vascular device including tissue which must be stored hydrated,
the endoluminal seal comprising an expandable material selected from the group consisting of hydrogels, sponges and foams optionally spray dried or chemically coupled to the interior of the endoluminal seal, wherein the expandable material is activated by exposure to a fluid or a foaming agent,
the compliant plug being shaped to be inserted into the vascular device to prevent fluid from passing beyond the tissue to reach the seal.

31. The plug of claim 30 formed of silicone or rubber.

32. The plug of claim 30 in a vascular device, further comprising mechanical means for compressing the exterior of the device against the inner plug.

33. The plug of claim 32 wherein the means of applying a mechanical pressure is a ratchet mechanism belt or other oversized compliant material belts.

34. A metal film or metal-polymer laminate for preventing exposure of an endoluminal seal for sealing an expandable vascular device including tissue which must be stored hydrated,
the endoluminal seal comprising an expandable material selected from the group consisting of hydrogels, sponges and foams optionally spray dried or chemically coupled to the interior of the endoluminal seal, wherein the expandable material is activated by exposure to a fluid or a foaming agent,
the laminate comprising a metallic film or a metallic film with a polymer laminate that acts as a barrier during the storage of the vascular device in fluid and is removable by peeling off the metal film or laminate along a pre-scored tear line.

35. The metal film or metal-polymer laminate of claim 34 further comprising full tabs to remove the metal film or barrier.

36. A packaging case for an endoluminal seal for sealing an expandable vascular device including tissue which must be stored hydrated,
the endoluminal seal comprising an expandable material selected from the group consisting of hydrogels, sponges and foams optionally spray dried or chemically coupled to the interior of the endoluminal seal, wherein the expandable material is activated by exposure to a fluid or a foaming agent,
the container having an upper and a lower compartment, which are not in fluid communication.

37. The packaging case of claim 36 comprising o-rings that fluidically separate the upper and lower compartments.

38. The packaging case of claim 36 further comprising a core that seals the upper tissue containing portion of the vascular device from the lower portion including the expandable seal.

39. The packaging case of claim 36 further comprising a polymeric material that is placed into the bottom compartment after insertion of the vascular device that creates a fluid seal between the upper tissue containing portion of the vascular device and the lower portion including the expandable seal.

40. A fluid absorbant material for placement within a vascular device to keep only the tissue portion hydrated.
